(19) United States
(12) Patent Application Publication
(10) Pub. No.: US 2024/0082236 A1
(43) Pub. Date: Mar. 14, 2024

(54) CANCER THERAPY

(71) Applicant: SURGE Therapeutics, Inc., Cambridge, MA (US)

(72) Inventor: Michael Solomon Goldberg, Brookline, MA (US)

(21) Appl. No.: 18/274,754

(22) PCT Filed: Jan. 28, 2022
(86) PCT No.: PCT/US22/14337

§ 371 Date: Jul. 28, 2023

Related U.S. Application Data
(60) Provisional application No. 63/143,836, filed on Jan. 30, 2021.

(51) Int. Cl. A61K 31/4745; A61K 31/395; A61K 31/407; A61K 31/41; A61K 31/728; A61K 31/765; A61K 38/20; A61K 45/06; A61P 35/00

(52) U.S. Cl. CPC A61K 31/4745; A61K 31/395; A61K 31/407; A61K 31/41; A61K 31/728; A61K 31/765; A61K 38/2013; A61K 38/208; A61K 45/06; A61P 35/00

(57) ABSTRACT

Provided herein are technologies suitable for the treatment of certain immunologically related disorders.

### CROSS REFERENCE TO RELATED APPLICATIONS

This application claims the benefit of U.S. Provisional Application No. 63/143,836 filed Jan. 30, 2021, the contents of which are hereby incorporated herein in their entirety.

### BACKGROUND

Cancer diagnosis and treatment each often involves partial or complete removal of one or more lymph nodes

### SUMMARY

The present disclosure provides certain insights relating to effective immunomodulatory therapies; in certain embodiments, provided technologies are particularly useful for the treatment of cancer.

Among other things, the present disclosure provides an insight that certain immunomodulatory compositions can provide effective impact when administered to a resection site from which part or all of at least one lymph node has been removed. For example, the present disclosure particularly describes use of certain biomaterial preparations as immunomodulatory compositions.

The present disclosure further appreciates that certain biomaterial preparations characterized by immunomodulatory properties (in some cases even without an immunomodulatory payload; see, for example, PCT/US20/31169 filed May 1, 2020; WO 2018/045058 and/or WO 2019/183216), may be particularly useful in the practice of technologies described herein.

Thus, among other things, the present disclosure provides an insight that certain biomaterial preparations may be particularly useful and/or may provide particular beneficial effects when administered in a certain fashion, e.g., as described herein.

In some embodiments, the present disclosure identifies the source of a problem with certain existing technologies including, for example, with certain systemic routes of anti-cancer therapeutics administration and/or delivery.

Moreover, the present disclosure provides an insight that unexpected benefits and/or advantages may be achieved by coupling administration of an immunomodulatory composition (e.g., a biomaterial preparation) as described herein with lymph node dissection. Among other things, lymph node dissection is often performed relatively early in the process of diagnosis and/or treatment of a cancer patient; early interventions can be particularly useful.

Furthermore, the present disclosure appreciates that immunomodulatory impact of administration as described herein may be particularly effective including because immune system component(s) (e.g., T cells, B cells, etc.) modified by a provided treatment can act in the lymph node itself and also can travel, e.g., via the lymphatic system and/or circulatory system, to other locations including to a primary tumor and/or to one or more metastases.

Still further, the present disclosure appreciates that various researchers have reported activation of metastatic lesions after lymph node resection (see, for example, Sukhbaatar et al., Cancer Sci. 110(2): 509-518, 2019 February, published online 2019 Jan. 17 as doi: 10.1111/cas.13898) and teaches that, in some embodiments, provided technologies may provide therapeutic benefit with respect to an existing tumor and/or metastasis and/or may reduce risk or incidence of negative impact(s) (e.g., activation of metastasis) that might otherwise be associated with the lymph node resection event.

In some embodiments, the present disclosure, among other things, provides methods of treating cancer that involves administration of an immunomodulatory composition at a lymph node dissection site in a cancer subject. In some embodiments, such a method comprises a step of administering to a target site in a cancer subject, a composition comprising an effective amount of an immunomodulatory composition, wherein the target site is or comprises a lymph node dissection site.

In some embodiments, an immunomodulatory composition that may be useful in accordance with the present disclosure may be characterized by its ability to induce an innate immune response. In some embodiments, an immunomodulatory composition that may be useful in accordance with the present disclosure may be characterized by its ability to induce an adaptive immune response. In some embodiments, an immunomodulatory composition that may be useful in accordance with the present disclosure may be characterized by its ability to induce an innate immune response and an adaptive immune response. In some embodiments, an immunomodulatory composition that may be useful in accordance with the present disclosure may be characterized by its ability to inhibit an immunosuppressive inflammation response.

In some embodiments, an immunomodulatory composition that may be useful in accordance with the present disclosure is or comprises a biomaterial preparation. In some embodiments, a biomaterial preparation that is included in an immunomodulatory composition described herein can be or comprise a polymeric biomaterial preparation. In some embodiments, such a polymeric biomaterial preparation may comprise one or more polymers (including, e.g., at least one polymer, at least two polymers, or more). In some embodiments, such a polymeric biomaterial preparation can comprise a thermo-responsive polymer. In some embodiments, such a thermo-responsive polymer can be or comprise a poloxamer. Additionally or alternatively, in some embodiments, a polymeric biomaterial preparation can comprise a carbohydrate polymer. For example, in some embodiments, a carbohydrate can comprise hyaluronic acid and/or chitosan or a modified chitosan. In some embodiments, a polymeric biomaterial preparation that may be useful in accordance with the present disclosure comprises a poloxamer at 7-12.5% (w/w), and one or both of a hyaluronic acid at 0.5-7% (w/w) and chitosan or a modified chitosan at 0.5-7% (w/w).

In some embodiments, an immunomodulatory composition that may be useful in accordance with the present disclosure can comprise a biomaterial preparation (e.g., as described herein) and at least one immunomodulatory payload. In some embodiments, such an immunomodulatory payload is or comprises a modulator of innate immunity. In some embodiments, such an immunomodulatory payload is or comprises a modulator of myeloid cell function. In some embodiments, such an immunomodulatory payload is or comprises a modulator of adaptive immunity. In some embodiments, such an immunomodulatory payload is or comprises a modulator of inflammation. In some embodiments, such an immunomodulatory payload is or comprises a Toll-like receptor (TLR) agonist (e.g., TLR7/8 agonist). In some embodiments, such an immunomodulatory payload is or comprises resiquimod. In some embodiments, such an immunomodulatory payload is or comprises a cyclooxygenase (COX) inhibitor. In some embodiments, such an immunomodulatory payload is or comprises a nonsteroidal anti-inflammatory drug (NSAID), e.g., but not limited to ketorolac. In some embodiments, such an immunomodulatory payload is or comprises an angiotensin II receptor inhibitor, e.g., valsartan. In some embodiments, such an immunomodulatory payload is or comprises a CXCR4 receptor antagonist, e.g., plerixafor. In some embodiments, such an immunomodulatory payload is or comprises an immunomodulatory cytokine, e.g., IL-2 or IL-12.

In some embodiments, technologies described herein may be amenable to a cancer subject who is a tumor resection subject.

In some embodiments, the present disclosure provides methods of treating cancer comprising removing at least one lymph node that is proximal to a tumor in a cancer subject, prior to administration of an immunomodulatory composition to the lymph node dissection site. In some embodiments, such removal of at least one lymph node can be performed intraoperatively during a tumor resection surgery. Alternatively, in some embodiments, such removal of at least one lymph node can be performed in a different operation from a tumor resection surgery.

In some embodiments, involving a tumor resection surgery, a second immunomodulatory composition can be administered at the tumor resection site. In some embodiments, a second immunomodulatory composition administered at a tumor resection site can be the same as an immunomodulatory composition administered at a lymph node dissection site in a cancer subject. Alternatively, in some embodiments, a second immunomodulatory composition administered at a tumor resection site can be different from an immunomodulatory composition administered at a lymph node dissection site.

In some embodiments involving a tumor resection surgery, such a tumor resection surgery may comprise removal of at least a portion of an organ comprising a tumor. In some embodiments, such a tumor resection surgery may further comprise anastomosis after the removal of at least a portion of the organ comprising the tumor.

In some embodiments, technologies described herein are amenable to a lymph node that can be or comprise a sentinel lymph node, a draining lymph node, an axillary lymph node, an inguinal lymph node, a femoral lymph node, a facial lymph node, a neck lymph node, a cervical lymph node, a supraclavicular lymph node, a subclavian lymph node, a pectoral lymph node, a mediastinal lymph node, a pelvic lymph node, a mesenteric lymph node, and/or a retroperitoneal lymph node. In some embodiments, such a lymph node can be or comprise a sentinel lymph node. In some embodiments, such a lymph node can be or comprise a draining lymph node.

In some embodiments, the present disclosure provides methods of treating cancer that comprise intraoperative administration of an immunomodulatory composition (e.g., as described herein) to a target site being or comprising a lymph node dissection site in a cancer subject undergoing a lymph node dissection surgery. In some embodiments, a lymph node dissection surgery may further comprise anastomosis.

In some embodiments, technologies described herein can be useful for treatment of cancer including, e.g., but not limited to a carcinoma, a sarcoma, a germ cell tumor, a blastoma, a lymphoma, a skin cancer, a melanoma, a pharyngeal head and neck cancer, a thyroid cancer, a brain cancer, a bladder cancer, a gastrointestinal tract cancer (e.g., a stomach cancer), a thoracic cancer, a lung cancer, a breast cancer, a colorectal cancer, a genitourinary cancer, a kidney cancer, a prostate cancer, a gynecologic cancer, a testicular cancer, an ovarian cancer, and or an uterine cancer. In some embodiments, technologies described herein may reduce the likelihood of developing one or more symptoms associated with lymphedema and/or lymphocele as compared to a lymph node dissection without administration of an immunomodulatory composition described herein.

In some embodiments, technologies described herein may reduce the likelihood of developing one or more metastatic lesions as compared to a lymph node dissection without administration of an immunomodulatory composition.

In one aspect, the present disclosure, among other things, provides improvements in a method of treating cancer by intraoperative administration of an immunomodulatory composition. In some embodiments, such improvement comprises administering an immunomodulatory composition at a lymph node dissection site rather than or in addition to at a tumor resection site. In some embodiments, such an immunomodulatory composition comprises or is a combination of a biomaterial preparation and an immunomodulatory payload. In some embodiments, such an immunomodulatory composition comprises a biomaterial preparation in the absence of an immunomodulatory payload.

### BRIEF DESCRIPTION OF THE DRAWINGS

FIG. 1 is a graphical representation of a survival analysis. The data is presented as a Kaplan-Meier survival curve, showing the survival of female BALB/cJ mice inoculated orthotopically with 4T1-Luc2 cells. These cells were allowed to create primary tumors. Those primary tumors and proximal lymph nodes were subsequently surgically resected. Immediately following primary tumor and proximal lymph node resection, an exemplary composition comprising a biomaterial preparation (a thermo-responsive polymer solution that forms a hydrogel at body temperature) and an immunomodulatory payload (resiquimod (R848)) was implanted at a target site at and/or near a lymph node resection site. Administration of compositions containing an immunomodulatory payload resulted in increased lifespan of animals when compared to compositions lacking an immunomodulatory payload.

### DEFINITIONS

Activator of adaptive immune response. The term “activator of adaptive immune response” refers to an agent in whose presence the adaptive immune system is more active than it is absent the agent. Without wishing to be bound by any particular theory, it is proposed that, in some embodiments, adaptive immune system activity (e.g., activation) can restore or enhance antitumor function, for example by neutralizing inhibitory immune checkpoints and/or by triggering co-stimulatory receptors, ultimately generating helper and/or effector T cell responses against immunogenic antigens expressed by cancer cells and producing memory B cell and/or T cell populations. Alternatively or additionally, in certain embodiments, adaptive immune system activity (e.g., activation) may involve modulation of an adaptive immune responses and/or leukocyte trafficking.

Activator of innate immune response. The term “activator of innate immune response” refers to an agent in whose presence the innate immune system is more active than it is absent the agent. Without wishing to be bound by any particular theory, it is proposed that, in some embodiments, innate immune system activity (e.g., activation) can stimulate the expression of molecules that initiate an inflammatory response and/or help to induce adaptive immune responses, for example leading to development of antigen-specific acquired immunity. In some embodiments, innate immune system activity (e.g., activation) can lead to cytokine production, immune cell proliferation and/or survival and/or improved T cell priming, for example by enhancing presentation of antigens and/or of co-stimulatory molecule expression by antigen-presenting cells.

Administer. The terms “administer,” “administering,” or “administration” refer to implanting, absorbing, ingesting, injecting, inhaling, or otherwise introducing a composition to a site on or in a subject as described herein.

Agonist. The term “agonist” refers to an agent that (i) increases or induces one or more effects of another agent; and/or (ii) increases or induces one or more biological events. In some embodiments, an agonist may increase level and/or activity or one or more agents that it targets. In various embodiments, agonists may be or include agents of various chemical class including, for example, small molecules, polypeptides, nucleic acids, carbohydrates, lipids, metals, and/or other entity that shows the relevant agonistic activity. An agonist may be direct (in which case it exerts its influence directly upon its target) or indirect (in which case it exerts its influence by other than binding to its target; e.g., by interacting with a regulator of the target, for example so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

Antagonist. The term “antagonist” refers to an agent that (i) decreases or suppresses one or more effects of another agent; and/or (ii) decreases or suppresses one or more biological events. In some embodiments, an antagonist may reduce level and/or activity or one or more agents that it targets. In various embodiments, antagonists may be or include agents of various chemical class including, for example, small molecules, polypeptides, nucleic acids, carbohydrates, lipids, metals, and/or other entity that shows the relevant antagonistic activity. An antagonist may be direct (in which case it exerts its influence directly upon its target) or indirect (in which case it exerts its influence by other than binding to its target; e.g., by interacting with a regulator of the target, for example so that level or activity of the target is altered). In some embodiments, an antagonist may be a receptor antagonist, e.g., a receptor ligand or drug that blocks or dampens a biological response by binding to and blocking a receptor rather than activating it like an agonist.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies. etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINs®; dual-affinity re-targeting (DART) agents; Fynomers®; shark single domain antibodies such as IgNAR; immune mobilixing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies, Trans-Bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Bioadhesive. The term “bioadhesive” refers to a biocompatible agent that can adhere to a target surface, e.g., a tissue surface. In some embodiments, a bioadhesive can adhere to a target surface, e.g., a tissue surface, and retain on the target surface, e.g., for a period of time. In some embodiments, a bioadhesive may be biodegradable. In some embodiments, a bioadhesive may be a natural agent, which may have been prepared or obtained, for example, by isolation or by synthesis; in some embodiments, a bioadhesive may be a non-natural agent, e.g., as may have been designed and/or manufactured by the hand of man (e.g., by processing, synthetic, and/or recombinant production, depending on the agent, as will be understood by those skilled in the art. In some particular embodiments, a bioadhesive may be or comprise a polymeric material, e.g., as may be comprised of or contain a plurality of monomers such as sugars. Certain exemplary bioadhesives include a variety of FDA-approved agents such as, for example, cyanoacrylates (Dermabond, 2-Octyl cyanoacrylate; Indermil, n-Butyl-2-cyanoacrylate; Histoacryl and Histoacryl Blue, n-Butyl-2-cyanoacrylate), albumin and glutaraldehyde (BioGlue™, bovine serum albumin and 10% glutaraldehyde), fibrin glue (Tisseel™, human pooled plasma fibrinogen and thrombin; Evicel™, human pooled plasma fibrinogen and thrombin; Vitagel™, autologous plasma fibrinogen and thrombin; Cryoseal™ system, autologous plasma fibrinogen and thrombin), gelatin and/or resorcinol cross-linked by formaldehyde and/or glutaraldehyde, polysaccharide-based adhesives (gelatin, collagen, dextran, chitosan, alginate), PEG, acrylates, polyamines, or urethane derivatives (isocyanate-terminated prepolymer, and/or combinations thereof. Other examples of bioadhesives that are known in the art, e.g., as described in Mehdizadeh and Yang “Design Strategies and Applications of Tissue Bioadhesives” Macromol Biosci 13:271-288 (2013), can be used for the purposes of the methods described herein. In some embodiments, a bioadhesive can be a degradable bioadhesive. Examples of such a degradable bioadhesive include, but are not limited to fibrin glues, gelatin-resorcinol-formaldehyde/glutaraldehyde glues, poly(ethylene glycol) (PEG)-based hydrogel adhesives, polysaccharide adhesives, polypeptide adhesives, polymeric adhesives, biomimetic bioadhesives, and ones described in Bhagat and Becker “Degradable Adhesives for Surgery and Tissue Engineering” Biomacromolecules 18: 3009-3039 (2017).

Biocompatible. The term “biocompatible” refers to a material that is substantially non-toxic in the in vivo environment of its intended use and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, and/or immunogenicity. A biocompatible structure or material, when introduced into a majority of patients, will not cause an undesirably adverse, long-lived, or escalating biological reaction or response and is distinguished from a mild, transient inflammation, which typically accompanies surgery or implantation of foreign objects into a living organism.

Biologic. The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

Biological sample. The term “biological sample” refers to a primary sample obtained from a biological source and/or, in some embodiments, to a sample derived therefrom (e.g., by processing). Those skilled in the art appreciate that biological samples may include or be selected from, for example, tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

Biomaterial. The term “biomaterial” refers to a biocompatible substance characterized in that it can be administered to a subject for a medical purpose (e.g., therapeutic, diagnostic) without eliciting an unacceptable (according to sound medical judgement) reaction. Biomaterials can be obtained or derived from nature or synthesized. In some embodiments, a biomaterial can be in a form of gel. In some embodiments, a biomaterial can be in an injectable format. For example, a biomaterial can comprise precursor components of a gel to be formed in situ (e.g., upon administration to a subject).

Cancer. The term “cancer” refers to a malignant neoplasm (Stedman's Medical Dictionary, 25th ed.; Hensyl ed.; Williams & Wilkins: Philadelphia, 1990). Of particular interest in the context of some embodiments of the present disclosure are cancers treated by cell killing and/or removal therapies (e.g., surgical resection and/or certain chemotherapeutic therapies such as cytotoxic therapies, etc). In some embodiments, a cancer that is treated in accordance with the present disclosure is one that has been surgically resected (i.e., for which at least one tumor has been surgically resected). In some embodiments, a cancer that is treated in accordance with the present disclosure is one for which resection is standard of care. In some embodiments, a cancer that is treated in accordance with the present disclosure is one that has metastasized. In certain embodiments, exemplary cancers may include one or more of acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); parathryroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget's disease of the vulva).

Chemotherapeutic agent. The term “chemotherapeutic agent” refers to a therapeutic agent known to be of use in chemotherapy for cancer.

Co-crystal. The term “co-crystal” refers to a crystalline structure composed of at least two components. In certain embodiments, a co-crystal contains a compound of interest (e.g., ones disclosed herein) and one or more other component(s), such as, for example, one or more atoms, ions, or molecules (e.g., solvent molecules). In certain embodiments, a co-crystal contains a compound of interest and one or more solvent molecules. In certain embodiments, a co-crystal contains a compound of interest and one or more acid or base.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those of ordinary skill in the art will also understand that when the term “comparable” is used in the context of comparison of two or more values, such values are comparable to one another such that the differences in values do not result in material differences in therapeutic outcomes, e.g., induction of anti-tumor immunity and/or incidence of tumor regrowth and/or metastasis. For example, in some embodiments, comparable release rates refer to values of such release rates within 15% over a period of 48 hours. In some embodiments, comparable release rates refer to values of such release rates within 20% over a period of 48 hours. In some embodiments, comparable release rates refer to values of such release rates within 15% over a period of 24 hours.

Condition, disease, or disorder. The terms “condition,” “disease,” and “disorder” are used interchangeably.

Corresponding to: As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Cross-linker. The term “cross-linker” refers to an agent that links one entity (e.g., one polymer chain) to another entity (e.g., another polymer chain). In some embodiments, linkage (i.e., the “cross-link”) between two entities is or comprises a covalent bond. In some embodiments, linkage between two entities is or comprises an ionic bond. In some embodiments, a cross-linker is a small molecule (e.g., dialdehydes or genipin) for inducing formation of a covalent bond between an aldehyde and an amino group. In some embodiments, a cross-linker comprises a photo-sensitive functional group. In some embodiments, a cross-linker comprises a pH-sensitive functional group. In some embodiments, a cross-linker comprises a thermal-sensitive functional group.

Effective amount. An “effective amount” is an amount sufficient to elicit a desired biological response, e.g., treating a condition from which a subject may be suffering. As will be appreciated by those of ordinary skill in this art, the effective amount of drug delivery composition may vary depending on such factors as the desired biological endpoint, the pharmacokinetics of the therapeutic agents in the composition, the condition being treated, and the age and health of the subject. In some embodiments, an amount may be effective for therapeutic treatment; alternatively or additionally, in some embodiments an amount may be effective for prophylactic treatment. For example, in treating cancer, an effective amount may prevent tumor regrowth, reduce the tumor burden, or stop the growth or spread of a tumor. Those skilled in the art will appreciate that an effective amount need not be contained in a single dosage form. Rather, administration of an effective amount may involve administration of a plurality of doses, potentially over time (e.g., according to a dosing regimen). For example, in some embodiments, an effective amount may be an amount administered in a dosing regimen that has been established, when administered to a relevant population, to achieve a particular result with statistical significance.

Hydrate. The term “hydrate”, as used herein, has its art-understood meaning and refers to an aggregate of a compound (which may, for example be a salt form of the compound) and one or more water molecules. Typically, the number of the water molecules contained in a hydrate of a compound is in a definite ratio to the number of the compound molecules in the hydrate. Therefore, a hydrate of a compound may be represented, for example, by the general formula R×x H2O, wherein R is the compound and x is a number greater than 0. A given compound may form more than one type of hydrate, including, e.g., monohydrates (x is 1), lower hydrates (x is a number greater than 0 and smaller than 1, e.g., hemihydrates (R×0.5 H2O)), and polyhydrates (x is a number greater than 1, e.g., dihydrates (R×2 H2O) and hexahydrates (R×6 H2O)).

Hydrogel. The term “hydrogel” has its art-understood meaning and refers to a material formed from a network of polymer chains that are hydrophilic, sometimes found as a colloidal gel in which an aqueous phase is the dispersion medium. In some embodiments, hydrogels are highly absorbent (e.g., they can absorb and/or retain over 90% water) natural or synthetic polymeric networks. In some embodiments, hydrogels possess a degree of flexibility similar to natural tissue, for example due to their significant water content.

Immunotherapy. The term “immunotherapy” refers to a therapeutic agent that promotes the treatment of a disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, small molecule inhibitors, and small molecule agonists. For example, useful immunotherapies may include, but are not limited to, inducers of type I interferon, interferons, stimulator of interferon genes (STING) agonists, TLR7/8 agonists, IL-15 superagonists, COX inhibitors (e.g., COX-1 inhibitors and/or COX-2 inhibitors), anti-PD-1 antibodies, anti-CD137 antibodies, and anti-CTLA-4 antibodies.

Implantable. The terms “implantable,” “implantation,” “implanting,” and “implant” refer to positioning a drug delivery composition at a specific location in a subject, such as within a tumor resection site or in a sentinel lymph node, and typically by general surgical methods.

Inhibit. The term “inhibit” or “inhibition” in the context of modulating level (e.g., expression and/or activity) of a target (e.g., p38 MAPK) is not limited to only total inhibition. Thus, in some embodiments, partial inhibition or relative reduction is included within the scope of the term “inhibition.” In some embodiments, the term refers to a reduction of the level (e.g., expression, and/or activity) of a target (e.g., p38 MAPK) to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target.

Inhibitor. As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

Inhibitor of a pro-inflammatory pathway. The term “inhibitor of a pro-inflammatory pathway” as used herein, in some embodiments, refers to an agent that prevents recruitment of immunosuppressive cells or prevents acute inflammation. Such acute inflammation and/or recruitment of immunosuppressive cells can occur after local trauma, including that which is caused by surgery. In some embodiments, an inhibitor of a pro-inflammatory pathway may inhibit, for example, an immune response that induces inflammation, including, e.g., production of pro-inflammatory cytokines (e.g., TNF-alpha, IL-1beta, and IL-6), increased activity and/or proliferation of Th1 cells, recruitment of myeloid cells, etc.

Isomers. It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Lymph node. As is known in the art, the term “lymph node” refers to components of the lymphatic system that are small structures, located throughout the body, through which lymph fluid flows. Lymph nodes are understood to filter certain substances from lymphatic fluid. Lymph nodes also can contain immune cells, for example that may participate in immune reactions throughout the body. In some embodiments, a lymph node may be or comprise a sentinel lymph node (i.e., a lymph node to which cancer cells are most likely to spread from a primary tumor).

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

Metastasis. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

Microparticle: The “microparticle” is used herein to refer to a discrete entity of small size, e.g., typically having a longest dimension that is shorter than about 1000 micrometers (μm), and often is shorter than 500 μm, or even 100 μm or less. In many embodiments, a microparticle may be characterized by a longest dimension between about 1 μm and about 100 μm, or between about 1 μm and about 500 μm, or between about 1 μm and 1000 μm. In many embodiments, a population of microparticles is characterized by an average size (e.g., longest dimension) that is below about 1000 μm, about 500 μm, about 100 μm, about 50 μm, about 40 μm, about 30 μm, about 20 μm, or about 10 μm and often above about 1 μm. In many embodiments, a microparticle may be substantially spherical (e.g., so that its longest dimension may be its diameter).

Modulator: As used herein, the term “modulator” may be or comprise an entity whose presence or level in a system in which an activity of interest is observed correlates with a change in level and/or nature of that activity as compared with that observed under otherwise comparable conditions when the modulator is absent. In some embodiments, a modulator is an activator or agonist, in that an activity of interest is increased in its presence as compared with that observed under otherwise comparable conditions when the modulator is absent. In some embodiments, a modulator is an antagonist or inhibitor, in that an activity of interest is reduced in its presence as compared with otherwise comparable conditions when the modulator is absent. In some embodiments, a modulator interacts directly with a target entity whose activity is of interest. In some embodiments, a modulator interacts indirectly (e.g., interacts with one or more entities that interacts and/or are associated with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Modulator of macrophage effector function. The term “modulator of macrophage effector function” refers to an agent that activates macrophage effector function or depletes immunosuppressive macrophages or macrophage-derived suppressor cells. Such potentiation can mobilize macrophage and myeloid components to destroy the tumor and its stroma, including the tumor vasculature. Macrophages can be induced to secrete antitumor cytokines and/or to perform phagocytosis, including antibody-dependent cellular phagocytosis.

Modulator of neutrophil function: As used interchangeably herein, the terms “modulator of neutrophils” and “modulator of neutrophil function” refer to a modulator of one or more biological functions and/or phenotypes of neutrophils. For example, in some embodiments, a modulator of neutrophil function can inhibit recruitment, survival, and/or proliferation of neutrophils. Additionally or alternatively, in some embodiments, a modulator of neutrophil function can modulate neutrophil-associated effector function, which may include but are not limited to, modulation of production and/or secretion of one or more immunomodulatory molecules (e.g., immunomodulatory cytokines and/or chemokines) and/or alter extracellular-matrix modifying capabilities of neutrophils. In some embodiments, a modulator of neutrophil function (e.g., ones described herein) may act on or target neutrophils only. In some embodiments, a modulator of neutrophil function (e.g., ones described herein) may act on neutrophils and at least one additional type of immune cells, e.g., other subsets of myeloid-derived suppressive cells (MDSCs), macrophages, and/or monocytes. One of ordinary skill in the art will appreciate that at least a subset of neutrophils may exhibit similar immune activities as one or more certain subsets of MDSCs and thus be considered as polymorphonuclear and/or granulocytic MDSCs (for example, as described in: Mehmeti-Ajradini et al., “Human G-MDSCs are neutrophils at distinct maturation stages promoting tumor growth in breast cancer” Life Science Alliance, Sep. 21, 2020; and Brandau et al., “A subset of mature neutrophils contains the strongest PMN-MDSC activity in blood and tissue of patients with head and neck cancer” The Journal of Immunology, May 1, 2020; the contents of each of which are incorporated herein by reference for purposes described herein).

Nanoparticle: As used herein, the term “nanoparticle” refers to a discrete entity of small size, e.g., typically having a longest dimension that is shorter than about 1000 nanometers (nm) and often is shorter than 500 nm, or even 100 nm or less. In many embodiments, a nanoparticle may be characterized by a longest dimension between about 1 nm and about 100 nm, or between about 1 μm and about 500 nm, or between about 1 nm and 1000 nm. In many embodiments, a population of microparticles is characterized by an average size (e.g., longest dimension) that is below about 1000 nm, about 500 nm, about 100 nm, about 50 nm, about 40 nm, about 30 nm, about 20 nm, or about 10 nm and often above about 1 nm. In many embodiments, a microparticle may be substantially spherical (e.g., so that its longest dimension may be its diameter). In some embodiments, a nanoparticle has a diameter of less than 100 nm as defined by the National Institutes of Health.

Neoplasm and tumor: The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a lipid, a metal, a nucleic acid, a polypeptide, a saccharide (e.g., a polysaccharide), small molecule, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, an payload may be or comprise an agent in isolated or pure form; in some embodiments, such agent may be in crude form.

Pharmaceutically acceptable salt. The term “pharmaceutically acceptable salt” refers to those salts which are, within the scope of sound medical judgment, suitable for use in contact with the tissues of, for example, humans and/or animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, the contents of which are incorporated herein by reference for purposes described herein. Pharmaceutically acceptable salts that may be utilized in accordance with certain embodiments of the present disclosure may include, for example, those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N+(C1-C4 alkyl)4- salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Poloxamer: As used herein, the term “poloxamer” refers to a polymer preparation of or comprising one or more poloxamers. In some embodiments, poloxamers in a polymer preparation may be unconjugated or unmodified, for example, which are typically triblock copolymers comprising a hydrophobic chain of polyoxypropylene (polypropylene glycol, PPG) flanked by two hydrophilic chains of polyoxyethylene (polyethylene glycol, PEG). In some embodiments, a polymer preparation of or comprising one or more poloxamer may be unfiltered (e.g., such a polymer preparation may contain impurities and/or relatively low molecular weight polymeric molecules, as compared to a comparable polymer preparation that is filtered). Examples of poloxamers include are not limited to, Poloxamer 124 (P124, also known as Pluronic L44 NF), Poloxamer 188 (P188, also known as Pluronic F68NF), Poloxamer 237 (P237, also known as Pluronic F 87 NF), Poloxamer 338 (P338, also known as Pluronic F108 NF), Poloxamer 407 (P407, also known as Pluronic F127 NF), and combinations thereof.

Polymer: The term “polymer” is given its ordinary meaning as used in the art, i.e., a molecular structure comprising one or more repeat units (monomers), connected by covalent bonds. The repeat units may all be identical, or, in some cases, there may be more than one type of repeat unit present within the polymer. In certain embodiments, a polymer is naturally occurring. In certain embodiments, a polymer is synthetic (i.e., not naturally occurring). In some embodiments, a polymer for use in accordance with the present disclosure is a polypeptide. In some embodiments, a polymer for use in accordance with the present disclosure is not a nucleic acid.

Polymer combination preparation: As used herein, the term “polymer combination preparation” refers to a polymeric biomaterial comprising at least two distinct polymer components. For example, in many embodiments, a polymer combination preparation described herein is a polymeric biomaterial comprising a first polymer component and a second first polymer component, wherein the first polymer component is or comprises at least one poloxamer, and the second polymer component is or comprises a polymer that is not poloxamer. In some embodiments, a polymer combination preparation described herein is a polymeric biomaterial in a precursor state, which may be, e.g., useful for administration to a subject. In some embodiments, a polymer combination preparation described herein is a polymeric biomaterial in a polymer network state.

Polymeric biomaterial: A “polymeric biomaterial”, as described herein, is a material that is or comprises at least one polymer or at least one polymeric moiety and is biocompatible. In many embodiments, a polymeric biomaterial is or includes at least one polymer; in some embodiments, a polymer may be or comprise a copolymer. In some embodiments, a polymeric biomaterial is or comprises a preparation of at least two distinct polymer components (e.g., a preparation containing poloxamer and a second polymer component that is not a poloxamer). Those skilled in the art will be aware that certain polymers may exist and/or be available in a variety of forms (e.g., length, molecular weight, charge, topography, surface chemistry, degree and/or type of modification such as alkylation, acylation, quaternization, hydroxyalkylation, carboxyalkylation, thiolation, phosphorylation, glycosylation, etc.); in some embodiments, a preparation of such polymers may include a specified level and/or distribution of such form or forms. Additionally or alternatively, those skilled in the art will appreciate that, in some embodiments, one or more immunomodulatory properties of a polymeric biomaterial may be tuned by its biomaterial property(ies), including, e.g., surface chemistry of a polymeric biomaterial (e.g., modulated by hydrophobicity and/or hydrophilicity portions of a polymeric biomaterial, chemical moieties, and/or charge characteristics) and/or topography of a polymeric biomaterial (e.g., modulated by size, shape, and/or surface texture), for example as described in Mariani et al. “Biomaterials: Foreign Bodies or Tuners for the Immune Response?” International Journal of Molecular Sciences, 2019, 20, 636.

Polymer network: The term “polymer network” is used herein to describe an assembly of polymer chains interacting with each other. In some embodiments, a polymer network forms a three-dimensional structure material. In some embodiments, a polymer network may be formed by linking polymer chains (“crosslinked polymer network”) using a crosslinker (e.g., as described herein). In some embodiments, a polymer network is transitioned from a precursor state when it is exposed to a temperature that is or above a critical gelation temperature, wherein the polymer network state has a viscosity materially above (e.g., at least 50% or above) that of the precursor state and the polymer network state comprises crosslinks not present in the precursor state. In some embodiments, a polymer network may be formed by non-covalent or non-ionic intermolecular association of polymer chains, e.g., through hydrogen bonding. In some embodiments, a polymer network may be formed by a combination of chemically crosslinking polymer chains and non-covalent or non-ionic intermolecular association of polymer chains.

Polymorph: The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting point, density, hardness, crystal shape, optical properties, electrical properties, stability, solubility, bioavailability, etc. Recrystallization solvent, rate of crystallization, storage temperature, and other factors may cause one crystal form to dominate a given preparation. Various polymorphs of a compound can typically be prepared by crystallization under different conditions.

Prodrug: The term “prodrug” refers to a form of an active compound that includes one or more cleavable group(s) that is/are removed by solvolysis or under physiological conditions, so that the active compound is released. Exemplary prodrug forms include, but are not limited to, choline ester derivatives and the like as well as N-alkylmorpholine esters and the like. In some embodiments, a prodrug may be an acid derivative, such as is known in the art, such as, for example, esters prepared by reaction of the parent acid with a suitable alcohol, amides prepared by reaction of the parent acid compound with a substituted or unsubstituted amine, acid anhydrides, or mixed anhydrides. Simple aliphatic or aromatic esters, amides, and anhydrides derived from acidic groups pendant on a compound of interest are particular examples of prodrug forms. In some cases, it may be desirable to prepare double ester-type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of a compound of interest.

Pro-inflammatory immune response: The term “pro-inflammatory immune response” as used herein refers to an immune response that induces inflammation, including, e.g., production of pro-inflammatory cytokines (e.g., TNF-alpha, IL-1beta, and IL-6), increased activity and/or proliferation of Th1 cells, recruitment of myeloid cells, etc. In some embodiments, a pro-inflammatory immune response may be or comprise one or both of acute inflammation and chronic inflammation.

Proliferative disease. A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

Prophylactically effective amount. A “prophylactically effective amount” is an amount sufficient to prevent (e.g., significantly delay onset or recurrence of one or more symptoms or characteristics of, for example so that it/they is/are not detected at a time point at which they would be expected absent administration of the amount) a condition. A prophylactically effective amount of a composition means an amount of therapeutic agent(s), alone or in combination with other agents, that provides a prophylactic benefit in the prevention of the condition. The term “prophylactically effective amount” can encompass an amount that improves overall prophylaxis or enhances the prophylactic efficacy of another prophylactic agent. Those skilled in the art will appreciate that a prophylactically effective amount need not be contained in a single dosage form. Rather, administration of an effective amount may involve administration of a plurality of doses, potentially over time (e.g., according to a dosing regimen).

Risk: as will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

Salt. As used herein, the term “salt” refers to any and all salts and encompasses pharmaceutically acceptable salts.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brochoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Small molecule. The term “small molecule” or “small molecule therapeutic” refers to a molecule, whether naturally occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, a small molecule is a therapeutically active agent such as a drug (e.g., a molecule approved by the U.S. Food and Drug Administration as provided in the Code of Federal Regulations (C.F.R.)). A small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, a small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460; drugs for veterinary use are listed by the FDA under 21 C.F.R. §§ 500 through 589, the contents of each of which are incorporated herein by reference for purposes described herein; such drugs are typically considered acceptable for use in accordance with the present disclosure.

Solvate. The term “solvate”, as used herein, has its art-understood meaning and refers to an aggregate of a compound (which may, for example, be a salt form of the compound) and one or more solvent atoms or molecules. In some embodiments, a solvate is a liquid. In some embodiments, a solvate is a solid form (e.g., a crystalline form). In some embodiments, a solid-form solvate is amenable to isolation. In some embodiments, association between solvent atom(s) and compound in a solvate is a non-covalent association. In some embodiments, such association is or comprises hydrogen bonding, van der Waals interactions, or combinations thereof. In some embodiments, a solvent whose atom(s) is/are included in a solvate may be or comprise one or more of water, methanol, ethanol, acetic acid, DMSO, THF, diethyl ether, and the like. Suitable solvates may be pharmaceutically acceptable solvates; in some particular embodiments, solvates are hydrates, ethanolates, or methanolates. In some embodiments, a solvate may be a stoichiometric solvate or a non-stoichiometric solvate.

Subject. A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a tumor resection subject, e.g., a subject who has recently undergone tumor resection. In some embodiments, a tumor resection subject is a subject who has undergone tumor resection in less than 72 hours (including, e.g., less than 48 hours, less than 24 hours, less than 12 hours, less than 6 hours, or lower), prior to receiving a drug delivery composition or device described herein. In some embodiments, a tumor resection subject is a subject who has undergone tumor resection in less than 48 hours, prior to receiving a drug delivery composition or device described herein. In some embodiments, a tumor resection subject is a subject who has undergone tumor resection in less than 24 hours, prior to receiving a drug delivery composition or device described herein. In some embodiments, a tumor resection subject is a subject who has undergone tumor resection in less than 12 hours, prior to receiving a drug delivery composition or device described herein.

Substantially: As used herein, the term “substantially” refers to the qualitative condition of exhibiting total or near-total extent or degree of a characteristic or property of interest. Those skilled in the art will understand that an agent of interest, if ever, achieves or avoids an absolute result, e.g., an agent of interest that indeed has zero effect on an immune response, e.g., inflammation. The term “substantially” is therefore used herein to capture the potential lack of absoluteness inherent in many biological and chemical effects.

Sustained: As used interchangeably herein, the term “sustained” or “extended” typically refers to prolonging an effect and/or a process over a desirable period of time. For example, in the context of sustained immunomodulation (e.g., in the presence of a composition or preparation as described and/or utilized herein), the term may refer to an immunomodulatory effect observed after administration of a particular immunomodulatory payload in the context of a composition comprising a biomaterial preparation and otherwise as described herein, as contrasted, for example, with administration of the same payload absent such biomaterial preparation and/or otherwise differently from administration described herein (e.g., not to a lymph node dissection site). Alternatively or additionally, the term “sustained” may refer to a characteristic of release of a payload from a composition as described herein, as contrasted with that observed, for example, by immediate release administration; in some embodiments, such sustained or extended release may occur on a timescale within a range of from about 30 minutes to several weeks or more. In some embodiments, the extent of sustained release or extended release can be characterized in vitro or in vivo. For example, in some embodiments, release kinetics can be tested in vitro by placing a preparation and/or composition described herein in an aqueous buffered solution (e.g., PBS at pH 7.4). In some embodiments, when a preparation and/or composition described herein is placed in an aqueous buffered solution (e.g., PBS at pH 7.4), less than 100% or lower (including, e.g., less than or equal to 90%, less than or equal to 80%, less than or equal to 70%, less than or equal to 50% or lower) of one or more agents of interest (e.g., payloads incorporated described herein and/or degradation or dissolution products and/or soluble components of polymer preparations described herein that modulate one or more aspects of an immune response, e.g., but not limited to innate immunity agonism) is released within 3 hours from a biomaterial. In some embodiments, release kinetics can be tested in vivo, for example by implanting a composition at a target site (e.g., mammary fat pad) of an animal subject (e.g., a mouse subject). In some embodiments, when a composition is implanted at a target site (e.g., mammary fat pad) of an animal subject (e.g., a mouse subject), less than or equal to 70% or lower (including, e.g., less than or equal to 60%, less than or equal to 50%, less than 40%, less than 30% or lower) of one or more agents of interest (e.g., payloads described herein and/or degradation or dissolution products and/or soluble components of polymer preparations described herein that modulate one or more aspects of an immune response, e.g., but not limited to innate immunity agonism) is released in vivo 8 hours after the implantation.

Targeted agent. The term “targeted agent”, when used in reference to an anticancer agent means one that blocks the growth and spread of cancer by interfering with specific molecules (“molecular targets”) that are involved in the growth, progression, and/or spread of cancer. Targeted agents are sometimes called “targeted cancer therapies,” “molecularly targeted drugs,” “molecularly targeted therapies,” or “precision medicines.” Targeted agents differ from traditional chemotherapy in that targeted agents typically act on specific molecular targets that are specifically associated with cancer, and/or with a particular tumor or tumor type, stage, etc., whereas many chemotherapeutic agents act on all rapidly dividing cells (e.g., whether or not the cells are cancerous). Targeted agents are deliberately chosen or designed to interact with their target, whereas many standard chemotherapies are identified because they kill cells.

Tautomers. The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

Test subject: As used herein, the term “test subject” refers to a subject to which technologies provided herein are applied for experimental investigation, e.g., to assess biomaterial degradation, and/or efficacy of compositions and/or preparations described herein in antitumor immunity. In some embodiments, a test subject may be a human subject or population of human subjects. For example, in some embodiments, a human test subject may be a normal healthy subject. In some embodiments, a human test subject may be a tumor resection subject. In some embodiments, a test subject may be a mammalian non-human animal or a population of mammalian non-human animals. Non-limiting examples of such mammalian non-human animals include mice, rats, dogs, pigs, rabbits, etc., which in some embodiments may be normal healthy subjects, while in some embodiments may be tumor resection subjects. In some embodiments, mammalian non-human animals may be transgenic or genetically engineered animals.

Therapeutic agent. The term “therapeutic agent” refers to an agent having one or more therapeutic properties that produce a desired, usually beneficial, effect. For example, a therapeutic agent may treat, ameliorate, and/or prevent (e.g., materially delay onset or progression of one or more symptoms or characteristics of) a disease, disorder, or condition. In some embodiments, a therapeutic agent may be or comprise a nucleic acid, a polypeptide, a small molecule, or a combination thereof; in some embodiments, a therapeutic agent may be subject to governmental regulation (e.g., pre-market approval assessment) as a biologic, a device, a drug, or a combination thereof.

Therapeutically effective amount. A “therapeutically effective amount” is an amount sufficient to provide a therapeutic benefit in the treatment of a condition, which therapeutic benefit may be or comprise, for example, reduction in frequency and/or severity, and/or delay of onset of one or more features or symptoms associated with the condition. A therapeutically effective amount means an amount of therapeutic agent(s), alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. Those skilled in the art will appreciate that a therapeutically effective amount need not be contained in a single dosage form. Rather, administration of an effective amount may involve administration of a plurality of doses, potentially over time (e.g., according to a dosing regimen, and particularly according to a dosing regimen that has been established, when applied to a relevant population, to provide an appropriate effect with a desired degree of statistical confidence).

Temperature-responsive: As used herein, the term “temperature-responsive”, in the context of a temperature-responsive polymer or biomaterial (e.g., polymeric biomaterial), refers to a polymer or biomaterial (e.g., polymeric biomaterial) that exhibits an instantaneous or discontinuous change in one or more of its properties at a critical temperature (e.g., a critical gelation temperature). For example, in some embodiments, one or more of such properties is or comprise a polymer's or biomaterial's solubility in a particular solvent. By way of example only, in some embodiments, a temperature-responsive polymer or biomaterial (e.g., polymeric biomaterial) is characterized in that it is a homogenous polymer solution or colloid that is stable below a critical temperature (e.g., a critical gelation temperature) and instantaneously form a polymer network (e.g., a hydrogel) when the critical temperature (e.g., critical gelation temperature) has been reached or exceeded. In some embodiments, a temperature-responsive polymer or biomaterial (e.g., polymeric biomaterial) may be temperature-reversible, e.g., in some embodiments where a polymer solution may instantaneously form a polymer network at a temperature of or above a critical gelation temperature, and such a resulting polymer network may instantaneously revert to a homogenous polymer solution when the temperature is reduced to below the critical gelation temperature.

Treat: The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein, e.g., cancer or tumor. In some embodiments, treatment may be administered after one or more signs or symptoms have developed or have been observed. Treatment may also be continued after symptoms have resolved, for example, to delay or prevent recurrence and/or spread.

Treatment. The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein. In some embodiments, treatment may be administered after one or more signs or symptoms have developed or have been observed. Treatment may also be continued after symptoms have resolved, for example, to delay or prevent recurrence and/or spread.

Tumor Site. The term “tumor site” may, in some embodiments, be a site in which at least a portion of a tumor is present or was present prior to resection. In some embodiments, a tumor site may still have the entirety of the tumor present. While in some embodiments, a tumor site may have part or all of the tumor removed, e.g., through tumor resection.

Tumor: The terms “tumor” and “neoplasm” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

Tumor removal: As used herein, the term “tumor removal” encompasses partial or complete removal of a tumor, which may be resulted from a cancer therapy, e.g., surgical resection. In some embodiments, tumor removal refers to physical removal of part or all of a tumor by surgery (i.e., “tumor resection”). In some embodiments, tumor removal may be resulted from a surgical tumor resection and an adjuvant therapy (e.g., chemotherapy, immunotherapy, and/or radiation therapy). In some embodiments, an adjuvant therapy may be administered after a surgical tumor resection, e.g., at least 24 hours or more after a surgical tumor resection.

Tumor resection subject: As used herein, the term “tumor resection subject” refers to a subject who is undergoing or has recently undergone a tumor resection procedure. In some embodiments, a tumor resection subject is a subject who has at least 70% or more (including, at least 80%, at least 90%, at least 95%, at least 98%, at least 99%, or higher (including 100%) of gross tumor mass removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection subject may be determined to have a negative resection margin (i.e., no cancer cells seen microscopically at the resection margin, e.g., based on histological assessment of tissues surrounding the tumor resection site). In some embodiments, a tumor resection subject may be determined to have a positive resection margin (i.e., cancer cells are seen microscopically at the resection margin, e.g., based on histological assessment of tissues surrounding the tumor resection site). In some embodiments, a tumor resection subject may have micrometastases and/or dormant disseminated cancer cells that can be driven to progress/proliferate by the physiologic response to surgery. In some embodiments, a tumor resection subject receives a composition (e.g., as described and/or utilized herein) immediately after the tumor resection procedure is performed (e.g., intraoperative administration). In some embodiments, a tumor resection subject receives a composition (e.g., as described and/or utilized herein) postoperatively within 24 hours or less, including, e.g., within 18 hours, within 12 hours, within 6 hours, within 3 hours, within 2 hours, within 1 hour, within 30 mins, or less.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant biomaterial (e.g., a variant polymer or a polymeric biomaterial comprising a variant polymer) may differ from a reference biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can retain the desired property(ies) and/or function(s) (e.g., immunomodulation and/or temperature-responsiveness) of the reference biomaterial. For example, a variant of an immunomodulatory biomaterial may differ from a reference immunomodulatory biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can act on an immune system (e.g., by stimulating innate immunity), e.g., when used in a method described herein. In some embodiments, a variant immunomodulatory biomaterial (e.g., a variant polymer or a polymeric biomaterial comprising a variant polymer) is characterized in that, when assessed at 24 hours after administration of such a variant immunomodulatory biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 60% or more (e.g., including, e.g., at least 70%, at least 80%, at least 90%, at least 95%, at least 98%, or up to 100%) of that observed when a reference biomaterial (e.g., a reference polymer or polymeric biomaterial) is administered at the target site. In some embodiments, a variant immunomodulatory biomaterial (e.g., a variant polymer or a polymeric biomaterial comprising a variant polymer) is characterized in that, when assessed at 24 hours after administration of such a variant biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 1.1-fold or more (e.g., including, e.g., at least 1.5-fold, at least 2-fold, at least 3-fold, at least 4-fold, at least 5-fold, or more) of that observed when a reference biomaterial (e.g., a reference polymeric biomaterial) is administered at the target site. In some embodiments, a variant biomaterial (e.g., a variant polymeric biomaterial) exhibits at least one physical characteristic that is different from that of a reference biomaterial (e.g., a reference polymeric biomaterial). For example, in some embodiments, a variant biomaterial (e.g., a variant polymeric biomaterial) can exhibit increased water solubility (e.g., at a physiological pH) as compared to that of a reference biomaterial (e.g., a reference polymeric biomaterial). In some embodiments, a variant has 10, 9, 8, 7, 6, 5, 4, 3, 2, or 1 structural modifications as compared with a reference. In some embodiments, a variant has a small number (e.g., fewer than 5, 4, 3, 2, or 1) number of structural modifications (e.g., alkylation, acylation, quaternization, hydroxyalkylation, carboxyalkylation, thiolation, phosphorylation, glycosylation, etc.). In some embodiments, a variant has not more than 5, 4, 3, 2, or 1 additions or deletions of chemical moieties, and in some embodiments has no additions or deletions, as compared with a reference. In some embodiments, a variant is an entity that can be generated from a reference by chemical manipulation. In some embodiments, a variant is an entity that can be generated through performance of a synthetic process substantially similar to (e.g., sharing a plurality of steps with) one that generates a reference.

### DETAILED DESCRIPTION OF CERTAIN EMBODIMENTS

The present disclosure, among other things, provides technologies, including, e.g., particular methods of administration that may be particularly useful and/or may provide particular beneficial effects, to certain patient populations e.g., as described herein.

Among other things, the present disclosure appreciates that partial or complete lymph node dissection is commonly utilized in the practice of cancer diagnosis and/or treatment and offers an insight that the lymph node resection bed may be a particularly attractive site for administration of certain immunomodulatory materials as described herein.

In some embodiments, the present disclosure utilizes materials that can be prepared and/or can adopt various different states (e.g., dry state, liquid state, gel state that, in some embodiments, may be or comprise a continuous gel and, in some embodiments, may be or comprise gel particles, or both).

In some embodiments, the present disclosure administers preparations in a liquid state; in some embodiments the present disclosure administers preparations in a pre-formed gel state, e.g., which may be characterized by flexible space-filling properties. In some such embodiments, the present disclosure utilizes preparations comprised of a relevant material in particulate form (e.g., so that the preparations comprise a plurality of particles, e.g., characterized by a size distribution and/or other parameters as described herein).

In some embodiments, the present disclosure utilizes temperature-responsive biomaterial preparations that, for example are able to transition from an injectable state to a polymer network state with material properties that provide beneficial effects, e.g., as described herein, without introduction of a cytotoxic crosslinking agent, e.g., UV radiation and/or chemical crosslinkers (e.g., small-molecule crosslinkers). Some such embodiments may be particularly useful for in situ formation of gelled materials, which may have various benefits relative to alternative technologies and may provide a solution to certain problems with such alternative technologies as identified herein. For example, the present disclosure identifies the source of a problem with various alternative technologies for in situ gelation, as many such technologies require treatments (e.g., exposure to UV radiation and/or to a chemical crosslinker, e.g., a small-molecule crosslinker) may have toxic or otherwise damaging effects for the recipient and/or for an agent that may be included in or with the material.

In some embodiments, immunomodulatory compositions (e.g., biomaterial preparations) for use in accordance with the present disclosure may demonstrate one or more immunomodulatory attributes, even in the absence of an immunomodulatory payload.

### I. Lymph Node Dissection

Lymph nodes are part of the lymphatic system, a network of vessels that carry lymph fluid throughout the body. An adult human typically has about 450 lymph nodes, which are grouped by location. For example, axillary nodes are under and around the armpit; inguinal nodes are in the groin; cervical nodes are in the head and neck area.

Lymph nodes are thought to be responsible for filtering lymph that passes through them. Lymph nodes contain lymphocytes (both T and B cells), which enter the lymph nodes from circulating blood.

Lymph node dissection (or resection) is a surgical procedure in which one or more lymph nodes is partially or fully removed from a subject, e.g., a subject who is suspected of or known to be suffering from cancer. Regional lymph node dissection involves removal of some lymph node(s) from a site (e.g., a site known or believed to be in the vicinity of a tumor); radical lymph node dissection involves removal of most or all of the lymph nodes from a site. As used herein, “lymph node dissection” and “lymph node resection” are used interchangeably to generally refer to a surgical procedure in which one or more lymph nodes is partially or fully removed from a subject. In certain embodiments, “lymph node dissection” may refer to removal of the entire cluster of lymph nodes from a site, while in certain embodiments, “lymph node dissection” may refer to removal of some lymph node(s), e.g., at least 1 or more, but not all, from a site.

In some embodiments, tissue removed during a lymph node dissection is processed (e.g., subjected to analysis under a microscope) to assess evidence of cancer and/or its type, stage, etc.

When a subject is known to have cancer and is undergoing surgical resection of part of all of one or more tumors, it is common to perform a lymph node dissection of nearby lymph nodes as part of the same surgical procedure in which a tumor is being resected. In fact, assessment of lymph nodes is central to the well-established “TNM” tumor staging system, which relies on the size of the primary tumor, and its status as having invaded (or not) nearby tissue (“T”), involvement of nearby lymph node (“N”), and presence of distant metastases (“M”) to classify tumors.

### II. Immunomodulatory Compositions

Among other things, the present disclosure provides insights that certain immunomodulatory compositions comprising biomaterial preparations may be particularly useful in the treatment of cancer when administered as described herein.

Certain exemplary immunomodulatory compositions comprising biomaterial preparations that may be useful in accordance with the present disclosure include those described in one or more of International Patent Application WO2018/045058 (see also U.S. Pat. Nos. 10,435,469, 10,413,612, 10,836,826 and US Patent Publication No US2020/00311930), International Patent Application WO2019/183216 (see also U.S. patent application Ser. No. 16/982,333), International Patent Application WO2020/223698, U.S. Patent Application No. 63/053,488 and/or U.S. Patent Application No. 63/108,861, the contents of each of which are incorporated herein by reference for purposes described herein.

In some embodiments, a utilized immunomodulatory composition can comprise a biomaterial preparation and a payload agent which, in many embodiments, is an immune system modulator as described herein (e.g., an immunomodulatory payload). Alternatively, in some embodiments, a utilized immunomodulatory composition comprising a biomaterial preparation may be substantially free of a known immunomodulatory payload(s).

In some embodiments, a biomaterial preparation described herein is characterized in that it forms a polymer network; without wishing to be bound by any particular theory, it is noted that, in some embodiments, such a network may act as a scaffold or depot for a payload (e.g., for an immunomodulatory payload) within an immunomodulatory composition.

In some embodiments, an immunomodulatory composition comprising a biomaterial preparation and a payload agent (e.g., in some embodiments an immunomodulatory payload) may perform as an extended release formulation, for example in that the payload is released from the composition more slowly (i.e., over a longer period of time) than is observed for an otherwise comparable composition lacking the biomaterial preparation (e.g., one or all polymer component(s) thereof).

In some embodiments, a biomaterial preparation for use as described herein comprises one or more polymers (e.g., ones described herein). In certain embodiments, a biomaterial preparation may comprise one or more positively charged polymers. In certain embodiments, a biomaterial preparation for use as described herein may comprise one or more negatively charged polymers. In certain embodiments, a relevant biomaterial preparation may comprise one or more neutral polymers. In certain embodiments, a relevant biomaterial preparation comprises one or more polymer components selected from: hyaluronic acid, alginate, chitosan, chitin, chondroitin sulfate, dextran, gelatin, collagen, starch, cellulose, polysaccharide, fibrin, poly-L-Lysine, methylcellulose, ethylene-vinyl acetate (EVA), poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyphosphazene, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, and/or combinations and/or derivatives thereof.

In many embodiments, a biomaterial preparation useful in accordance with the present disclosure is characterized by in situ gelation at a target site in the absence of crosslinking treatments (e.g., introduction of UV radiation and/or chemical crosslinkers) that may have toxic or otherwise damaging effects for the recipient and/or for a payload that is included in or with a biomaterial.

In some embodiments, a biomaterial preparation useful in accordance with the present is temperature-responsive, so that, e.g., its gelation (e.g., its transition from a liquid state to a gelled state) can occur upon exposure to a particular temperature. In many such embodiments, exposure to body temperature (e.g., by application to a site) is sufficient to trigger such gelation; in some embodiments, additional warmth may be applied. By way of example only, in some embodiments, a temperature-responsive biomaterial preparation as described herein is characterized in that it transitions from a precursor state (e.g., a liquid state or an injectable state) to a polymer network state that has a viscosity and/or storage modulus materially above that of the precursor state (e.g., a more viscous state or a hydrogel) when such a biomaterial preparation is exposed to a temperature at or above critical gelation temperature (CGT) for the biomaterial preparation. In some embodiments, a CGT for a biomaterial preparation for use in accordance with the present disclosure is at least 10° C. or higher, including e.g. at least 10° C., at least 11° C., at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., 33° C., at least 34° C., at least 35° C., at least 36° C., at least 37° C., at least 38° C., at least 39° C., at least 40° C., or higher. In some embodiments, a CGT for a relevant biomaterial preparation is about 10° C. to about 15° C. In some embodiments, a CGT for a relevant biomaterial preparation is about 12° C. to about 17° C. In some embodiments, a CGT for a relevant biomaterial preparation is about 14° C. to about 19° C. In some embodiments, a CGT for a relevant biomaterial preparation is about 16° C. to about 21° C. In some embodiments, a CGT for a relevant biomaterial preparation is about 18° C. to about 23° C. In some embodiments, a CGT for a relevant biomaterial preparation is about 20° C. to about 25° C. In some embodiments, a CGT for a relevant biomaterial preparation is about 22° C. to about 27° C. In some embodiments, a CGT for a relevant biomaterial preparation is about 24° C. to about 29° C. In some embodiments, a CGT for a relevant biomaterial preparation is about 26° C. to about 31° C. In some embodiments, a CGT for a relevant biomaterial preparation is about 28° C. to about 33° C. In some embodiments, a CGT for a relevant biomaterial preparation is about 30° C. to about 35° C. In some embodiments, a CGT for a relevant biomaterial preparation is about 32° C. to about 37° C. In some embodiments, a CGT for a relevant biomaterial preparation is about 34° C. to about 39° C. In some embodiments, a CGT for a relevant biomaterial preparation is about 35° C. to about 39° C. In some embodiments, a CGT for a relevant biomaterial preparation is at or near physiological temperature of a subject (e.g., a human subject) receiving such a biomaterial preparation.

In some embodiments, a biomaterial preparation for use in accordance with the present disclosure is temperature-reversible. For example, in some embodiments, a relevant biomaterial preparation is characterized in that it transitions from a precursor state (e.g., a liquid state or an injectable state) to a polymer network state that has a viscosity and/or storage modulus materially above that of the precursor state (e.g., a more viscous state or a hydrogel) when such a biomaterial preparation is exposed to a temperature at or above critical gelation temperature (CGT) for the biomaterial preparation; and it may revert from the polymer network state to a state that has a viscosity and/or storage modulus materially lower than that of the polymer network state (e.g., a liquid state or original state of a relevant biomaterial preparation).

In some embodiments, a biomaterial preparation described herein does not comprise a chemical crosslinker. Those of skill in the art will appreciate that, in some embodiments, a chemical crosslinker is characterized in that it facilitates formation of covalent crosslinks between polymer chains. In some embodiments, a chemical crosslinker is or comprises a small-molecule crosslinker, which can be derived from a natural source or synthesized. Non-limiting examples of small-molecule crosslinkers include genipin, dialdehyde, glutaraldehyde, glyoxal, diisocyanate, glutaric acid, succinic acid, adipic acid, acrylic acid, diacrylate, etc.). In some embodiments, a chemical crosslinker may involve crosslinking using thiols (e.g., EXTRACEL®, HYSTEM®), methacrylates, hexadecylamides (e.g., HYMOVIS®), and/or tyramines (e.g., CORGEL®). In some embodiments, a chemical crosslinker may involve crosslinking using formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, and/or genipin (see, e.g., Khunmanee et al. “Crosslinking method of hyaluronic-based hydrogel for biomedical applications” J Tissue Eng. 8: 1-16 (2017)). Accordingly, in some embodiments, crosslinks that form during the transition from a precursor state to a polymer network state do comprise covalent crosslinks.

In some embodiments, a biomaterial preparation (e.g., a temperature responsive biomaterial preparation) for use as described herein is or comprises a poloxamer or a variant thereof. In some embodiments, a poloxamer or a variant thereof is present in a relevant biomaterial preparation at a concentration of no more than 12.5% (w/w) (including, e.g., no more than 12% (w/w), no more than 11.5% (w/w), no more than 11% (w/w), no more than 10.5% (w/w), no more than 10% (w/w), no more than 9.5% (w/w), no more than 9% (w/w), no more than 8% (w/w)), no more than 7% (w/w), no more than 6% (w/w), or no more than 5% (w/w). In some embodiments, a poloxamer or a variant thereof is present in a relevant biomaterial preparation at a concentration of 5% (w/w) to 12.5% (w/w), or 8% (w/w) to 12.5% (w/w), or 5% (w/w) to 10% (w/w), or 8% (w/w) to 10% (w/w).

Among other things, the present disclosure appreciates that hydrogel technologies comprising certain crosslinking technologies may produce toxic by-products and/or may adversely affect stability and/or efficacy of agent(s) (e.g., therapeutic agents) that may be combined with polymer biomaterials before or during crosslinking.

Alternatively or additionally, the present disclosure appreciates that, in some embodiments, particular advantages can be achieved by administering component(s) of a biomaterial preparation so that an immunomodulatory composition as described herein is formed during and/or upon administration as compared with pre-forming (e.g., by cross-linking) a polymer biomaterial prior to introducing it into a subject. For example, administration of a pre-formed biomaterial requires proportionate incisions and/or surgical intervention to facilitate administration. In some embodiments, for example, the present disclosure appreciates that such pre-forming generates a material with a defined size and/or structure, which may restrict options for administration, as the dimensions of the pre-formed material may differ from those of a resection cavity. In some embodiments, an immunomodulatory composition comprising a biomaterial preparation in a form of a hydrogel may be formed during and/or upon administration. In some embodiments, an immunomodulatory composition administered to a target site may comprise a pre-formed hydrogel biomaterial preparation.

In some embodiments, the present disclosure appreciates that a biomaterial preparation upon administration to a target site described herein may form a viscous solution. For example, in some embodiments, a liquid biomaterial preparation may be introduced to a target site so that an immunomodulatory composition as described herein in a form of a viscous solution (e.g., a solution with a viscosity of about 5,000 to 15,000 centipoise at body temperature, e.g., a solution with a viscosity of about 10,000 centipoise at body temperature) is formed upon administration to a target site.

In some embodiments, the present disclosure appreciates that a biomaterial preparation that is useful for administration to a target site described herein may be a viscous liquid solution, which can be substantially retained at the target site upon administration for a certain period of time. In some embodiments, such a viscous liquid biomaterial preparation has a viscosity that is low enough to be injectable (e.g., through a syringe tip or a catheter and/or a syringe needle) but is high enough to be substantially retained at a target site upon administration for a certain period of time. In some embodiments, such a viscous liquid biomaterial preparation may have a viscosity of about 500 to 10,000 centipoise at room temperature. In some embodiments, such a viscous liquid biomaterial preparation may have a viscosity of about 500 to 3,000 centipoise at room temperature. In some embodiments, such a viscous liquid biomaterial preparation may have a viscosity of about 1,000 to 8,000 centipoise at room temperature. In some embodiments, such a viscous liquid biomaterial preparation may have a viscosity of about 2,000 to 6,000 centipoise at room temperature. In some embodiments, such a viscous liquid biomaterial preparation may have a viscosity of about 3,000 to 7,000 centipoise at room temperature. In some embodiments, such a viscous liquid biomaterial preparation may have a viscosity of about 4,000 to 8,000 centipoise at room temperature. In some embodiments, such a viscous liquid biomaterial preparation may have a viscosity of about 5,000 to 9,000 centipoise at room temperature. In some embodiments, such a viscous liquid biomaterial preparation may have a viscosity of about 6,000 to 10,000 centipoise at room temperature.

In some embodiments, the present disclosure appreciates that there may be a viscosity constraint and/or limit on injectability of a liquid biomaterial preparation. For example, in some embodiments, an injectable biomaterial preparation may be characterized by a viscosity amenable to loading and controlled release through a needle of a set gauge (e.g., a needle with a gauge of between 14 and 20, e.g., a needle with a gauge of 16-18). Alternatively, in some embodiments, an injectable biomaterial preparation may be characterized by a viscosity amenable to loading and controlled release through a syringe tip of a set diameter (i.e., without a connected needle, or with a catheter). In some embodiments, a biomaterial preparation included in an immunomodulatory composition (e.g., as described herein) loaded into a syringe may further comprise a plasticizer.

The present disclosure provides technologies, including particular biomaterial preparations, and methods of administration, that permit interventions that may be less invasive than implantation and/or less toxic than systemic administration. In some such embodiments, preparations with improved administration characteristics may be administered in a liquid state; in some embodiments they may be administered in a pre-formed gel state characterized by flexible space-filling properties; in some embodiments they may be administered subcutaneously; in some embodiments they may function as a proximal depot for sustained release of immunomodulatory payloads (e.g., ones described herein); in some embodiments they may permit reprogramming of tissues such as sentinel and/or draining lymph nodes; in some embodiments they may be administered prior to or contemporaneously with a tumor resection surgery; in some embodiments, they may be administered ipsilaterally when compared to a tumor resection site and/or primary tumor site; in some embodiments, they may be administered contralaterally when compared to a tumor resection site and/or primary tumor site; in some embodiments, they may be administered to patients who have metastatic, disseminated, and/or recurrent cancers. In some such embodiments, provided preparations are comprised of a relevant material in particulate form (e.g., so that the preparations comprise a plurality of particles, e.g., characterized by a size distribution and/or other parameters as described herein).

Among other things, in some embodiments, the present disclosure provides methods of administration and methods of treatment comprising certain temperature-responsive biomaterial preparations that, for example are able to transition from an injectable state to another state with material properties that provide beneficial effects, e.g., as described herein, without introduction of a cytotoxic crosslinking agent, e.g., UV radiation and/or small-molecule crosslinkers. Some such embodiments thus provide valuable technologies for in situ formation of gelled materials, which technologies have various benefits relative to alternative technologies, and provide a solution to certain problems with such alternative technologies as identified herein. For example, the present disclosure identifies the source of a problem with various alternative technologies for in situ gelation, as many such technologies require treatments (e.g., exposure to UV radiation and/or to a small-molecule crosslinker), that may have toxic or otherwise damaging effects for the recipient and/or for an agent that may be included in or with the material.

In some embodiments, provided methods of administration and/or methods of treatment utilizing biomaterial preparations (e.g., in some embodiments temperature-responsive biomaterial preparations as described in U.S. Provisional Application 63/108,861 filed on Nov. 2, 2020, and/or U.S. Provisional Application 63/053,488 filed on Jul. 17, 2020; the contents of each of which are incorporated herein by reference for purposes described herein) may demonstrate one or more immunomodulatory attributes, even in the absence of an immunomodulatory payload. For example, in some embodiments, provided biomaterial preparations coupled with methods of administration that induce acute inflammatory responses may promote innate immunity upon administration to a target site in subject in need thereof (e.g., a patient diagnosed with cancer).

A. Exemplary Biomaterials

(i) Exemplary Temperature-Responsive Biomaterial Preparations

In some embodiments, a temperature-responsive biomaterial preparation that is useful in accordance with the present disclosure may comprise one or more poloxamers. Poloxamer is typically a block copolymer comprising a hydrophobic chain of polyoxypropylene (e.g., polypropylene glycol, PPG, and/or poly(propylene oxide), PPO) flanked by two hydrophilic chains of polyoxyethylene (e.g., polyethylene glycol, PEG, and/or poly(ethylene oxide), PEO). Poloxamers are known by the trade names Synperonics, Pluronic, and/or Kolliphor. Generally, poloxamers are non-ionic surfactants, which in some embodiments may have a good solubilizing capacity, low toxicity, and/or high compatibility with cells, body fluids, and a wide range of chemicals.

In some embodiments, a poloxamer for use in accordance with the present disclosure may be a poloxamer known in the art. For example, as will be understood by a skilled person in the art, poloxamers are commonly named with the letter P (for poloxamer) followed by three digits: the first two digits multiplied by 100 give the approximate molecular mass of the polyoxypropylene chain, and the last digit multiplied by 10 gives the percentage polyoxyethylene content. By way of example only, P407 refers to a poloxamer with a polyoxypropylene molecular mass of 4000 g/mol and a 70% polyoxyethylene content). A skilled person in the art will also understand that for the Pluronic and Synperonic tradenames, coding of such poloxamers starts with a letter to define its physical form at room temperature (e.g., L=liquid, P=paste, F=flake (solid)) followed by two or three digits, wherein the first digit (two digits in a three-digit number) in the numerical designation, multiplied by 300, indicates the approximate molecular weight of the polyoxypropylene chain; and the last digit, multiplied by 10, gives the percentage polyoxyethylene content. By way of example only, L61 refers to a liquid preparation of poloxamer with a polyoxypropylene molecular mass of 1800 g/mol and a 10% polyoxyethylene content. In addition, as will be apparent to a skilled artisan, poloxamer 181 (P181) is equivalent to Pluronic L61 and Synperonic PE/L61.

In some embodiments, a poloxamer that may be included in a biomaterial preparation described herein may be or comprise Poloxamer 124 (e.g., Pluronic L44 NF), Poloxamer 188 (e.g., Pluronic F68NF), Poloxamer 181 (e.g., Pluronic L61), Poloxamer 182 (e.g., Pluronic L62), Poloxamer 184 (e.g., Pluronic L64), Poloxamer 237 (e.g., Pluronic F87 NF), Poloxamer 338 (e.g., Pluronic F108 NF), Poloxamer 331 (e.g., Pluronic L101), Poloxamer 407 (e.g., Pluronic F127 NF), or combinations thereof. In some embodiments, a biomaterial preparation for use in accordance with the present disclosure can comprise at least two or more different poloxamers. Additional poloxamers as described in Table 1 of Russo and Villa “Poloxamer Hydrogels for Biomedical Applications” Pharmaceutics (2019) 11(12):671, the contents of which are incorporated herein by reference for purposes described herein, may be also useful for biomaterial preparations described herein.

In some embodiments, a provided temperature-responsive biomaterial preparation comprises a first polymer component (e.g., a poloxamer) and a second polymer component that is not a poloxamer. In some embodiments, a second polymer component may be present in a relevant biomaterial preparation at a concentration of no more than 10% (w/w), including, e.g., at a concentration of 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, a second polymer component may be present in a biomaterial preparation for use in accordance with the present disclosure at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), or higher. In some embodiments, a second polymer component in a relevant biomaterial preparation may be present at a concentration of 0.1% (w/w) to 10% (w/w), or 0.1% (w/w) to 8% (w/w), or 0.1% (w/w) to 5% (w/w), or 1% (w/w) to 5% (w/w).

In some embodiments, a second polymer component included in a biomaterial preparation for use in accordance with the present disclosure may be or comprise a carbohydrate polymer, e.g., a polymer that is or comprises a carbohydrate, e.g., a carbohydrate backbone, including, e.g., but not limited to hyaluronic acid, chitosan, and/or variants thereof.

In some embodiments, the present disclosure appreciates, among other things, that certain conventional preparations that are or comprise a poloxamer and that are used to form a hydrogel typically utilize such that are or comprise a poloxamer (e.g., Poloxamer 407 (P407)) at a minimum concentration of 16-20% (w/w). The present disclosure identifies the source of a problem with such conventional preparations, including that they may have certain disadvantages for administration to subjects, including, e.g., high solution viscosity that makes it less ideal for injection, and/or tissue irritation due to high concentrations of poloxamers. Moreover, the present disclosure demonstrates that it is possible to develop useful preparations with materially lower concentration(s) of such poloxamers.

For example, in some embodiments, the present disclosure provides an insight that certain poloxamers, e.g., Poloxamer 407 (P407), which have been typically used at a minimum concentration of 16-20% (w/w) to form a hydrogel, can form a useful temperature-responsive biomaterial at concentrations lower than 16% (w/w), including, e.g., lower than 14% (w/w), lower than 12% (w/w), lower than 10% (w/w), or lower than 8% (w/w), when combined with one or more biocompatible polymers. In some embodiments, such biocompatible polymers may be or comprise a polymer that is not temperature-responsive, e.g., in some embodiments which may be or comprise hyaluronic acid and/or chitosan or modified chitosan.

In some embodiments, a biomaterial preparation comprising a poloxamer at a concentration of 12.5% (w/w) or below and at least one additional polymer that is not poloxamer may be immunomodulatory itself in the absence of an immunomodulatory payload. For example, in some embodiments, such a biomaterial preparation comprising a poloxamer at a concentration of 12.5% (w/w) or below and at least one carbohydrate polymer (e.g., hyaluronic acid or chitosan) may promote innate immunity upon administration to a target site in subject in need thereof (e.g., tumor resection subjects).

(ii) Exemplary Hyaluronic Acid-Containing Preparations

In some embodiments, a biomaterial preparation for use in accordance with the present disclosure is or comprises hyaluronic acid or a variant thereof.

Hyaluronic acid (HA), also known as hyaluronan or hyaluronate, is a non-sulfated member of a class of polymers known as glycosaminoglycans (GAG) that is widely distributed in body tissues. HA is found as an extracellular matrix component of tissue that forms a pericellular coat on the surfaces of cells.

HA is a polysaccharide (which in some embodiments may be present as a salt, e.g., a sodium salt, a potassium salt, and/or a calcium salt) having a molecular formula of (C₁₄H₂₁NO₁₁)ₙ where n can vary according to the source, isolation procedure, and/or method of determination.

In some embodiments, HA that may be useful in accordance with the present disclosure can be isolated or derived from a natural source; those skilled in the art will be aware that HA is available in a variety of natural sources. For example, in some embodiments, HA can be isolated or derived from, e.g., human umbilical cord, rooster combs, and/or connective matrices of vertebrate organisms. In some embodiments, HA can be isolated or derived from a capsular component of bacteria such as Streptococci. See, e.g., Kendall et al, (1937), Biochem. Biophys. Acta, 279, 401-405. In some embodiments, HA and/or variants thereof can be produced via microbial fermentation. In some embodiments, HA and/or variants thereof may be a recombinant HA or variants thereof, for example, produced using Gram-positive and/or Gram-negative bacteria as a host, including, e.g., but not limited to Bacillus sp., Lactococcus lactis, Agrobacterium sp., and/or Escherichia coli.

In some embodiments, a hyaluronic acid and/or variants thereof may be present in a relevant biomaterial preparation at a concentration of no more than 20% (w/w), including, e.g., at a concentration of 20% (w/w), 19% (w/w), 18% (w/w), 17% (w/w), 16% (w/w), 15% (w/w), 14% (w/w), 13% (w/w) 12% (w/w), 11% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, a hyaluronic acid and/or variants thereof may be present in a relevant biomaterial preparation at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), at least 19% (w/w), or higher. In some embodiments, a hyaluronic acid and/or variants thereof may be present in a relevant biomaterial preparation at a concentration of 0.1% (w/w) to 20% (w/w), 0.1% (w/w) to 18% (w/w), 0.1% (w/w) to 16% (w/w), 0.1% (w/w) to 14% (w/w), 0.10% (w/w) to 12% (w/w), 0.10% (w/w) to 10% (w/w), or 0.10% (w/w) to 8% (w/w), or 0.10% (w/w) to 5% (w/w). As will be appreciated by a skilled artisan, concentrations of hyaluronic acid and/or variants thereof in biomaterial preparations may be varied, e.g., depending on their molecular weights and/or desired mechanical characteristics (e.g., storage moduli, viscosities, etc.). For example, in some embodiments, low molecular weight hyaluronic acid (e.g., as described herein) and/or variants thereof may be present in biomaterial preparations at higher concentrations, e.g., in some embodiments at a concentration of 3% (w/w) to 7% (w/w). In some embodiments, high molecular weight hyaluronic acids (e.g., as described herein) and/or variants thereof may be present in biomaterial preparations at lower concentrations, e.g., in some embodiments at a concentration of 1% (w/w) to 3% (w/w).

In some embodiments, a relevant biomaterial preparation described herein may be a hyaluronic acid viscous solution. For example, in some embodiments, a hyaluronic acid viscous solution may have hyaluronic acid and/or variants thereof at a concentration of at least 0.1% (w/w) or higher, including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 2% (w/w), at least 3% (w/w), at least 4% (w/w), at least 5% (w/w), at least 6% (w/w), at least 7% (w/w), at least 8% (w/w), at least 9% (w/w), at least 10% (w/w), at least 11%, at least 12%, at least 13%, at least 14%, at least 15%, at least 16%, at least 17%, at least 18%, at least 19%, at least 20%, or higher. In some embodiments, a hyaluronic acid viscous solution may have hyaluronic acid and/or variants thereof at a concentration of no more than 20% (w/w), including, e.g., no more than 15%, no more than 14% (w/w), no more than 13% (w/w), no more than 12% (w/w), no more than 11% (w/w), no more than 10% (w/w), no more than 9% (w/w), no more than 8% (w/w), no more than 7% (w/w), no more than 6% (w/w), no more than 5% (w/w), no more than 4% (w/w), no more than 3% (w/w), no more than 2% (w/w), no more than 1% (w/w), no more than 0.5% (w/w), or lower. In some embodiments, combinations of the above-identified concentration ranges are possible. For example, in some embodiments, a hyaluronic acid viscous solution may have hyaluronic acid and/or variants thereof at a concentration of about 0.1% (w/w) to about 20% (w/w), or about 0.1% (w/w) to about 15% (w/w), or about 0.5% (w/w) to about 10% (w/w). In some embodiments, a high molecular weight hyaluronic acid (e.g., as described herein) and/or variants thereof may be present in a hyaluronic acid viscous solution at a concentration of 0.1% (w/w) to about 5% (w/w), or about 0.5% (w/w) to about 3% (w/w). In some embodiments, a low molecular weight hyaluronic acid (e.g., as described herein) and/or variants thereof may be present in a hyaluronic acid viscous solution at a concentration of 1.5% (w/w) to about 10% (w/w), or about 3% (w/w) to about 7% (w/w). As will be appreciated by a skilled artisan, higher concentrations of hyaluronic acid and/or variants thereof may be used if it is present in a biomaterial preparation as primary polymer or a substantially single polymer.

In some embodiments, HA or variants thereof that may be included in a biomaterial preparation for use in accordance with the present disclosure can have a low molecular weight, for example, an average molecular weight of 500 kDa or less, including, e.g., 450 kDa, 400 kDa, 350 kDa, 300 kDa, 250 kDa, 200 kDa, 150 kDa, 100 kDa, 50 kDa, or less. In some embodiments, HA or variants thereof that may be included in a relevant biomaterial preparation may have an average molecular weight of about 100 kDa to about 150 kDa. In some embodiments, HA or variants thereof that may be included in a relevant biomaterial preparation may have an average molecular weight of about 300 kDa to about 400 kDa.

In some embodiments, HA or variants thereof that may be included in a biomaterial preparation for use in accordance with the present disclosure can have a high molecular weight, for example, an average molecular weight of greater than 500 kDa or higher, including, e.g., 550 kDa, 600 kDa, 650 kDa, 700 kDa, 750 kDa, 800 kDa, 850 kDa, 900 kDa, 950 kDa, 1 MDa, 1.1 MDa, 1.2 MDa, 1.3 MDa, 1.4 MDa, 1.5 MDa, 1.6 MDa, 1.7 MDa, 1.8 MDa, 1.9 MDa, 2 MDa, 2.5 MDa, 3 MDa, 3.5 MDa, 4 MDa, 4.5 MDa, or higher. In some embodiments, HA or variants thereof that may be useful in accordance with the present disclosure may have an average molecular weight of about 600 kDa to about 900 kDa. In some embodiments, HA or variants thereof that may be useful in accordance with the present disclosure may have an average molecular weight of about 1 MDa to about 3 MDa.

In some embodiments, a biomaterial preparation for use in accordance with the present disclosure comprises a hyaluronic acid variant. In some embodiments, a hyaluronic acid variant is water-soluble. In some embodiments, a hyaluronic acid variant may be a chemically modified hyaluronic acid, e.g., in some embodiments, hyaluronic acid is esterified. Examples of chemical modifications to hyaluronic acid include, but are not limited to, addition of thiol, haloacetate, butanediol, diglycidyl, ether, dihydrazide, aldehyde, glycan, and/or tyramine functional groups. Additional hyaluronic acid modifications and variants are known in the art. See e.g., Highley et al., “Recent advances in hyaluronic acid hydrogels for biomedical applications” Curr Opin Biotechnol (2016) August 40:35-40; Burdick & Prestwich, “Hyaluronic acid hydrogels for biomedical applications” Advanced Materials (2011); Prestwhich, “Hyaluronic acid-based clinical biomaterials derived for cell and molecule delivery in regenerative medicine” J. Control Release (2011) October 30; 155(2): 193-199; the contents of each of which are incorporated herein by reference for purposes described herein.

(iii) Exemplary Chitosan-Containing Preparations

In some embodiments, a carbohydrate polymer included in a biomaterial preparation for use in accordance with the present disclosure may be or comprise chitosan or a variant thereof. Examples of chitosan and/or variants thereof that can be included in a biomaterial preparation described herein include, but are not limited to chitosan, chitosan salts (e.g., chitosan HCl, chitosan chloride, chitosan lactate, chitosan acetate, chitosan glutamate), alkyl chitosan, aromatic chitosan, carboxyalkyl chitosan (e.g., carboxymethyl chitosan), hydroxyalkyl chitosan (e.g., hydroxypropyl chitosan, hydroxyethyl chitosan), aminoalkyl chitosan, acylated chitosan, phosphorylated chitosan, thiolated chitosan, quaternary ammonium chitosan (e.g., N-(2-hydroxyl) propyl-3-trimethyl ammonium chitosan chloride), guanidinyl chitosan, chitosan oligosaccharide, glycated chitosan (e.g., N-dihydrogalactochitosan), and variants or combinations thereof. In some embodiments, a carbohydrate polymer included in a relevant biomaterial preparation comprising poloxamer (e.g., as described herein) may be or comprise carboalkyl chitosan (e.g., carboxymethyl chitosan).

Those skilled in the art will appreciate that, in some cases, chitosan and/or variants thereof can be produced by deacetylation of chitin. In some embodiments, chitosan or variants thereof included in a biomaterial preparation comprising poloxamer (e.g., as described herein) is characterized by degree of deacetylation (i.e., percent of acetyl groups removed) of at least 70% or above, including, e.g., at least 75%, at least 80%, at least 85%, at least 90%, at least 95%, or higher (including up to 100%). In some embodiments, a chitosan or variants thereof is characterized by degree of deacetylation of no more than 99%, no more than 95%, no more than 90%, no more than 85%, no more than 80%, no more than 75% or lower. Combinations of the above-mentioned ranges are also possible. For example, a chitosan or variants thereof may be characterized by degree of deacetylation of 80%-95%, 70%-95%, or 75%-90%. As will be recognized by one of those skilled in the art, degree of deacetylation (% DA) can be determined by various methods known in the art, e.g., in some cases, by NMR spectroscopy,

In some embodiments, chitosan and/or variants thereof may be present in a relevant biomaterial preparation at a concentration of no more than 20% (w/w), including, e.g., at a concentration of 20% (w/w), 19% (w/w), 18% (w/w), 17% (w/w), 16% (w/w), 15% (w/w), 14% (w/w), 13% (w/w) 12% (w/w), 110% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, chitosan and/or variants thereof may be present in a relevant biomaterial preparation at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), at least 19% (w/w), or higher. In some embodiments, chitosan and/or variants thereof may be present in a relevant biomaterial preparation at a concentration of 0.1% (w/w) to 20% (w/w), 0.10% (w/w) to 18% (w/w), 0.10% (w/w) to 16% (w/w), 0.10% (w/w) to 14% (w/w), 0.10% (w/w) to 12% (w/w), 0.1% (w/w) to 10% (w/w), or 0.1% (w/w) to 8% (w/w), or 0.1% (w/w) to 5% (w/w). As will be appreciated by a skilled artisan, concentrations of chitosan and/or variants thereof in biomaterial preparations may be varied, e.g., depending on their molecular weights and/or desired mechanical characteristics (e.g., storage moduli, viscosities, etc.). For example, in some embodiments, low molecular weight chitosan (e.g., in some embodiments <500 kDa) and/or variants thereof may be present in biomaterial preparations at higher concentrations, e.g., in some embodiments at a concentration of 3% (w/w) to 7% (w/w). In some embodiments, high molecular weight chitosan (e.g., in some embodiments >500 kDa) and/or variants thereof may be present in biomaterial preparations at lower concentrations, e.g., in some embodiments at a concentration of 1% (w/w) to 3% (w/w).

In some embodiments, a relevant biomaterial preparation described herein may be a chitosan viscous solution. For example, in some embodiments, a chitosan solution may have chitosan and/or variants thereof at a concentration of at least 0.1% (w/w) or higher, including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 2% (w/w), at least 3% (w/w), at least 4% (w/w), at least 5% (w/w), at least 6% (w/w), at least 7% (w/w), at least 8% (w/w), at least 9% (w/w), at least 10% (w/w), at least 11%, at least 12%, at least 13%, at least 14%, at least 15%, at least 16%, at least 17%, at least 18%, at least 19%, at least 20%, or higher. In some embodiments, a chitosan viscous solution may have chitosan and/or variants thereof at a concentration of no more than 20% (w/w), including, e.g., no more than 15%, no more than 14% (w/w), no more than 13% (w/w), no more than 12% (w/w), no more than 11% (w/w), no more than 10% (w/w), no more than 9% (w/w), no more than 8% (w/w), no more than 7% (w/w), no more than 6% (w/w), no more than 5% (w/w), no more than 4% (w/w), no more than 3% (w/w), no more than 2% (w/w), no more than 1% (w/w), no more than 0.5% (w/w), or lower. In some embodiments, combinations of the above-identified concentration ranges are possible. For example, in some embodiments, a chitosan viscous solution may have chitosan and/or variants thereof at a concentration of about 0.1% (w/w) to about 20% (w/w), or about 0.10% (w/w) to about 15% (w/w), or about 0.5% (w/w) to about 10% (w/w). In some embodiments, a high molecular weight chitosan (e.g., in some embodiments >300 kDa or in some embodiments >500 kDa) and/or variants thereof may be present in a chitosan viscous solution at a concentration of 0.1% (w/w) to about 5% (w/w), or about 0.5% (w/w) to about 3% (w/w). In some embodiments, a low molecular weight chitosan (e.g., in some embodiments <500 kDa or in some embodiments <300 kDa) and/or variants thereof may be present in a chitosan viscous solution at a concentration of 1.5% (w/w) to about 10% (w/w), or about 3% (w/w) to about 7% (w/w). As will be appreciated by a skilled artisan, higher concentrations of chitosan and/or variants thereof may be used if it is present in a biomaterial preparation as primary polymer or a substantially single polymer.

In some embodiments, chitosan or variants thereof included in a biomaterial preparation described herein may have an average molecular weight of at least 5 kDa or higher, including, e.g., at least 10 kDa or higher, including, e.g., at least 20 kDa, at least 30 kDa, at least 40 kDa, at least 50 kDa, at least 60 kDa, at least 70 kDa, at least 80 kDa, at least 90 kDa, at least 100 kDa, at least 110 kDa, at least 120 kDa, at least 130 kDa, at least 140 kDa, at least 150 kDa, at least 160 kDa, at least 170 kDa, at least 180 kDa, at least 190 kDa, at least 200 kDa, at least 210 kDa, at least 220 kDa, at least 230 kDa, at least 240 kDa, at least 250 kDa, at least 260 kDa, at least 270 kDa, at least 280 kDa, at least 290 kDa, at least 300 kDa, at least 350 kDa, at least 400 kDa, at least 500 kDa, at least 600 kDa, at least 700 kDa, or higher. In some embodiments, chitosan or variants thereof included in a biomaterial preparation described herein may have an average molecular weight of no more than 750 kDa or lower, including, e.g., no more than 700 kDa, no more than 600 kDa, no more than 500 kDa, no more than 400 kDa, no more than 300 kDa, no more than 200 kDa, no more than 100 kDa, no more than 50 kDa, or lower.

Combinations of the above-mentioned ranges are also possible. For example, in some embodiments, chitosan or variants thereof included in a biomaterial preparation described herein is characterized by an average molecular weight of 10 kDa to 700 kDa, or 20 kDa to 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a biomaterial preparation described herein is characterized by an average molecular weight of 20 kDa to 700 kDa, or 30 kDa to 500 kDa. As noted herein, an average molecular weight may be a number average molecular weight, weight average molecular weight, or peak average molecular weight.

In some embodiments, chitosan or variants thereof included in a biomaterial preparation described herein is characterized by a molecular weight distribution in a range of 10 kDa to 700 kDa, or 20 kDa or 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a biomaterial preparation described herein is characterized by a molecular weight distribution in a range of 20 kDa to 700 kDa, or 30 kDa to 500 kDa.

In some embodiments, chitosan or variants thereof that may be included in a biomaterial preparation for use in accordance with the present disclosure can have a low molecular weight, for example, an average molecular weight of 500 kDa or less, including, e.g., 450 kDa, 400 kDa, 350 kDa, 300 kDa, 250 kDa, 200 kDa, 190 kDa, 180 kDa, 170 kDa, 160 kDa, 150 kDa, 140 kDa, 130 kDa, 120 kDa, 110 kDa, 100 kDa, 90 kDa, 80 kDa, 70 kDa, 60 kDa, 50 kDa, 40 kDa, 30 kDa, 20 kDa, or less. In some embodiments, chitosan or variants thereof that may be included in a biomaterial preparation for use in accordance with the present disclosure can have a low molecular weight of 350 kDa or 300 kDa, or lower. In some embodiments, chitosan or variants thereof that may be included in a relevant biomaterial preparation may have an average molecular weight of about 50 kDa to about 500 kDa. In some embodiments, chitosan or variants thereof that may be included in a relevant biomaterial preparation may have an average molecular weight of about 100 kDa to about 350 kDa or about 100 kDa to about 300 kDa.

In some embodiments, chitosan or variants thereof that may be included in a biomaterial preparation for use in accordance with the present disclosure can have a high molecular weight, for example, an average molecular weight of greater than 300 kDa or higher, including, e.g., 310 kDa, 320 kDa, 330 kDa, 340 kDa, 350 kDa, 360 kDa, 370 kDa, 380 kDa, 390 kDa, 400 kDa, 410 kDa, 420 kDa, 430 kDa, 440 kDa, 450 kDa, 460 kDa, 470 kDa, 480 kDa, 490 kDa, 500 kDa, 510 kDa, 520 kDa, 530 kDa, 540 kDa, 550 kDa, 560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, or higher. In some embodiments, chitosan or variants thereof that may be included in a biomaterial preparation for use in accordance with the present disclosure can have a high molecular weight of greater than 500 kDa or higher. In some embodiments, chitosan or variants thereof that may be useful in accordance with the present disclosure may have an average molecular weight of about 310 kDa to about 380 kDa. In some embodiments, chitosan or variants thereof that may be useful in accordance with the present disclosure may have an average molecular weight of about 350 kDa to about 500 kDa. In some embodiments, chitosan or variants thereof that may be useful in accordance with the present disclosure may have an average molecular weight of about 500 kDa to about 750 kDa.

In some embodiments, chitosan or variants thereof included in a biomaterial preparation described herein may be characterized by a viscosity of no more than 3500 mPa·s or lower, including, e.g., no more than 3000 mPa·s, no more than 2500 mPa·s, no more than 2000 mPa·s, no more than 1500 mPa·s, no more than 1000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, chitosan or variants thereof may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1000 mPa·s, at least 1500 mPa·s, at least 2000 mPa·s, at least 2500 mPa·s, or higher. Combinations of the above-mentioned ranges are also possible. For example, in some embodiments, such a viscous polymer solution of or comprising chitosan or variants thereof may be characterized by a viscosity of 5 mPa·s to 3000 mPa·s, or 5 mPa·s to 300 mPa·s, 5 mPa·s to 200 mPa·s, or 20 mPa·s to 200 mPa·s, or 5 mPa·s to 20 mPa·s. In some embodiments, viscosity of chitosan or variants thereof described herein is measured at 1% in 1% acetic acid at 20° C.

In some embodiments, a biomaterial preparation described herein comprises at least one or more (e.g., 1, 2, 3 or more) chitosan and/or variants thereof (including, e.g., modified chitosan and/or salts of chitosan or modified chitosan such as a chloride salt or a glutamate salt). For example, in some embodiments, chitosan and/or variants thereof (including, e.g., modified chitosan and/or salts of chitosan or modified chitosan such as a chloride salt or a glutamate salt) may be characterized by degree of deacetylation of 70%-95%, or 75%-90%, or 80%-95%, or greater than 90%. In some embodiments, chitosan and/or variants thereof (including, e.g., modified chitosan and/or salts of chitosan or modified chitosan such as a chloride salt or a glutamate salt) may be characterized by an average molecular weight of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., modified chitosan and/or salts of chitosan or modified chitosan such as a chloride salt or a glutamate salt) may be characterized by a molecular weight distribution in the range of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., salts thereof such as a chloride salt or a glutamate salt) may be characterized by a viscosity ranging from 5 to 3000 mPa·s, or 5 to 300 mPa·s, or 20 to 200 mPa·s. In some embodiments, such chitosan and/or variants thereof (including, e.g., salts thereof such as a chloride salt or a glutamate salt) may be or comprise PROTASAN™ UltraPure chitosan chloride and/or chitosan glutamate salt (e.g., obtained from NovoMatrix®, which is a business unit of FMC Health and Nutrition (now a part of Du Pont; Product No. CL 113, CL 114, CL 213, CL 214, G 113, G 213, G 214). In some embodiments, such chitosan and/or variants thereof (including, e.g., salts thereof such as a chloride salt or a glutamate salt) may be or comprise chitosan, chitosan oligomers, and/or variants thereof (including, e.g., Chitosan HCl, carboxymethyl chitosan, chitosan lactate, chitosan acetate), e.g., obtained from Heppe Medical Chitosan GMBH (e.g., Chitoceuticals® or Chitoscience®).

In some embodiments, chitosan or variants thereof included in a biomaterial preparation described herein is or comprises carboxyalkyl chitosan (e.g., carboxymethyl chitosan) that is characterized by at least one or all of the following characteristics: (1) degree of deacetylation of 80%-95%; (ii) an average molecular weight of 30 kDa to 500 kDa; or a molecular weight distribution of 30 kDa to 500 kDa; and (iii) a viscosity ranging from 5 to 300 mPa·s.

In some embodiments, chitosan or variants thereof included in a biomaterial preparation described herein is or comprises a variant of chitosan (e.g., as described herein). In some embodiments, such a variant of chitosan may include chemical modification(s) of one or more chemical moieties, e.g., hydroxyl and/or amino groups, of the chitosan chains. In some embodiments, such a variant of chitosan is or comprises a modified chitosan such as, e.g., but not limited to a glycated chitosan (e.g., chitosan modified by addition of one or more monosaccharide or oligosaccharide side chains to one or more of its free amino groups). Exemplary glycated chitosan that are useful herein include, e.g., but are not limited to ones described in U.S. Pat. Nos. 5,747,475, 6,756,363, WO 2013/109732, US 2018/0312611, and US 2019/0002594, the contents of each of which are incorporated herein by reference for the purposes described herein.

In some embodiments, chitosan or variants thereof included in a biomaterial preparation described herein is or comprises chitosan conjugated with a polymer that increases its solubility in aqueous environment (e.g., a hydrophilic polymer such as polyethylene glycol).

In some embodiments, chitosan or variants thereof included in a biomaterial preparation described herein is or comprises thiolated chitosan. Various modifications to chitosans, e.g., but not limited to carboxylation, PEGylation, galactosylation (or other glycations), and/or thiolation are known in the art, e.g., as described in Ahmadi et al. Res Pharm Sci., 10(1): 1-16 (2015), the contents of which are incorporated herein by reference for the purposes described herein. Those skilled in the art reading the present disclosure will appreciate that other modified chitosans can be useful for a particular application in which a method is being practiced.

In some embodiments, a biomaterial preparation described herein may be or comprise a polymer combination preparation as described in the Applicants Copending Applications U.S. Prov Application No. 63/053,488 filed Jul. 17, 2020, and U.S. Prov Application No. 63/108,861 filed on Nov. 2, 2020 (the contents of each of which are incorporated herein by reference for purposes described herein). For example, in some embodiments, a biomaterial preparation described herein may comprise poloxamer (e.g., P407) and hyaluronic acid. In some embodiments, a biomaterial preparation described herein may comprise poloxamer (e.g., P407), hyaluronic acid, and chitosan or a variant thereof.

(iv) Exemplary Characteristics and/or Properties of Described Immunomodulatory Compositions

In certain embodiments, an immunomodulatory composition comprises a biomaterial preparation that can extend the release of a immunomodulatory payload when delivered to a target site (e.g., at and/or near a lymph node resection site) relative to administration of the same a immunomodulatory payload in solution. In certain embodiments, a biomaterial preparation (e.g., a polymeric biomaterial) extends the release of a immunomodulatory payload at a target site (e.g., at and/or near a lymph node resection site) relative to administration of the same immunomodulatory payload in solution by at least 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, 2 weeks, 3 weeks, or 4 weeks. In some embodiments, a biomaterial preparation extends release of a immunomodulatory payload so that, when assessed at a specified time point after administration, more immunomodulatory payload is present at a target site (e.g., at and/or near a lymph node resection site) relative to the levels observed when the immunomodulatory payload is administered in solution. For example, in some embodiments, when assessed at 24 hours after administration, the amount of an immunomodulatory payload released to and present at a target site (e.g., at and/or near a lymph node resection site) is at least 30% more (including, e.g., at least 40%, at least 50%, at least 60%, at least 70%, at least 80%, at least 90%, or more) than that is observed when the immunomodulatory payload is administered in solution. In some embodiments, when assessed at 48 hours after administration, the amount of an immunomodulatory payload released to and present at a target site (e.g., at and/or near a lymph node resection site) is at least 30% more (including, e.g., at least 40%, at least 50%, at least 60%, at least 70%, at least 80%, at least 90%, or more) than that is observed when the immunomodulatory payload is administered in solution. In some embodiments, when assessed at 3 days after administration, the amount of an immunomodulatory payload released to and present at a target site (e.g., at and/or near a lymph node resection site) is at least 30% more (including, e.g., at least 40%, at least 50%, at least 60%, at least 70%, at least 80%, at least 90%, or more) than that is observed when the immunomodulatory payload is administered in solution. In some embodiments, when assessed at 5 days after administration, the amount of an immunomodulatory payload released to and present at a target site (e.g., at and/or near a lymph node resection site) is at least 30% more (including, e.g., at least 40%, at least 50%, at least 60%, at least 70%, at least 80%, at least 90%, or more) than that is observed when the immunomodulatory payload is administered in solution.

In some embodiments, compositions comprising a biomaterial preparation described herein (e.g., a polymeric biomaterial in a precursor state or in a polymer network state) can be characterized by a viscosity of no more than 10,000 mPa·s or lower, including, e.g., no more than 9000 mPa·s, no more than 8000 mPa·s, no more than 7000 mPa·s, no more than 6000 mPa·s, no more than 5000 mPa·s, no more than 4000 mPa·s, no more than 3500 mPa·s, no more than 3000 mPa·s, no more than 2500 mPa·s, no more than 2000 mPa·s, no more than 1500 mPa·s, no more than 1000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, compositions comprising a biomaterial preparation described herein (e.g., a polymeric biomaterial in a precursor state or in a polymer network state) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1000 mPa·s, at least 1500 mPa·s, at least 2000 mPa·s, at least 2500 mPa·s, at least 3000 mPa·s, at least 4000 mPa·s, at least 5000 mPa·s, at least 6000 mPa·s, at least 7000 mPa·s, at least 8000 mPa·s, at least 9000 mPa·s, or higher. Combinations of the above-mentioned ranges are also possible. For example, in some embodiments, compositions comprising a biomaterial preparation described herein (e.g., a polymeric biomaterial in a precursor state or in a polymer network state) may be characterized by a viscosity of 5 mPa·s to 10,000 mPa·s, or 10 mPa·s to 5000 mPa·s, or 5 mPa·s to 200 mPa·s, or 20 mPa·s to 100 mPa·s, or 5 mPa·s to 20 mPa·s. One skilled in the art reading the present disclosure will appreciate that, in some cases, viscosity of a composition comprising a biomaterial preparation described herein may be selected or adjusted based on, e.g., administration routes (e.g., injection vs. implantation), injection volume and/or time, and/or impact duration of immunomodulation. As will be also understood by one skilled in the art, viscosity of a biomaterial preparation depends on, e.g., temperature and concentration of the polymer in a testing sample. In some embodiments, viscosity of compositions comprising a biomaterial preparation described herein may be measured at 20° C., e.g., with a shear rate of 1000 s⁻¹.

In some embodiments, when a biomaterial preparation described herein is in a polymer network state, such a polymer network state may be characterized by a storage modulus of at least 100 Pa, at least 200 Pa, at least 300 Pa, at least 400 Pa, at least 500 Pa, at least 600 Pa, at least 700 Pa, at least 800 Pa, at least 900 Pa, at least 1000 Pa, at least 1100 Pa, at least 1200 Pa, at least 1300 Pa, at least 1400 Pa, at least 1500 Pa, at least 1600 Pa, at least 1700 Pa, at least 1800 Pa, at least 1900 Pa, at least 2000 Pa, at least 2100 Pa, at least 2200 Pa, at least 2300 Pa, at least 2400 Pa, at least 2500 Pa, at least 2600 Pa, at least 2700 Pa, at least 2800 Pa, at least 2900 Pa, at least 3000 Pa, at least 3500 Pa, at least 4000 Pa, at least 4500 Pa, at least 5000 Pa, at least 6000 Pa, at least 7000 Pa, at least 8000 Pa, at least 9000 Pa, or higher. In some embodiments, a biomaterial preparation in a polymer network may be characterized by a storage modulus of no more than 10 kPa, no more than 9 kPa, no more than 8 kPa, no more than 7 kPa, no more than 6 kPa, or lower. Combinations of the above-mentioned ranges are also possible. For example, in some embodiments, a biomaterial preparation in a polymer network may be characterized by a storage modulus of 100 Pa to 10 kPa, or 200 Pa to 5000 Pa, or 300 Pa to 2500 Pa, or 500 Pa to 2500 Pa or 100 Pa to 500 Pa. One of those skilled in the art will appreciate that various rheological characterization methods (e.g., as described in Weng et al., “Rheological Characterization of in situ Crosslinkable Hydrogels Formulated from Oxidized Dextran and N-Carboxyethyl Chitosan” Biomacromolecules, 8: 1109-1115 (2007)) can be used to measure storage modulus of a material, and that, in some cases, storage modulus of a material may be measured with a rheometer and/or dynamic mechanical analysis (DMA). One of those skilled in the art will also appreciate that rheological characterization can vary with surrounding condition, e.g., temperature and/or pH.

As will be appreciated by those skilled in the art reading the present disclosure, biomaterial preparations useful as described herein are typically biocompatible. In some embodiments, biomaterial preparations useful as described herein are biodegradable in vivo. In some embodiments, at least one polymer component in biomaterial preparations for use in accordance with the present disclosure may be biodegradable in vivo. In some embodiments, at least one polymer component in relevant biomaterial preparations may be resistant to biodegradation (e.g., via enzymatic and/or oxidative mechanisms). In some embodiments, at least one polymer component in relevant biomaterial preparations may be chemically oxidized. Accordingly, in some embodiments, biomaterial preparations are able to be degraded, chemically and/or biologically, within a physiological environment, such as within a subject's body, e.g., at a target site of a subject. Those skilled in the art will appreciate, reading the present disclosure, that degradation rates of biomaterial preparations for use in accordance with the present disclosure may vary, e.g., based on selection of polymer component(s) and their material properties, and/or concentrations thereof (e.g., as described herein). For example, the half-life of relevant biomaterial preparations (the time at which 50% of a biomaterial preparation is degraded into monomers and/or other non-polymeric moieties) may be on the order of days, weeks, months, or years. In some embodiments, biomaterial preparations described herein may be biologically degraded, e.g., by enzymatic activity or cellular machinery, for example, through exposure to a lysozyme (e.g., having relatively low pH), or by simple hydrolysis. In some cases, relevant biomaterial preparations may be broken down into monomers (e.g., polymer monomers) and/or non-polymeric moieties that are non-toxic to cells. As will be understood by one of those skilled in the art, a relevant biomaterial preparation has a longer residence time at a target site (e.g., at and/or near a lymph node resection site) upon administration if such a relevant biomaterial preparation has a slower in vivo degradation rate.

In some embodiments, a biomaterial preparation for use in accordance with the present disclosure is characterized in that, when assessed in vivo by administering to a target site (e.g., at and/or near a lymph node resection site) in a test subject (e.g., as described herein), at least 10% or more, including, e.g., at least 20%, at least 30%, at least 40%, at least 50%, at least 60%, at least 70%, at least 80%, at least 90%, or more, of such a relevant biomaterial preparation in a polymer network state remains at the target site in vivo 2 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a relevant biomaterial preparation in a polymer network state remains at a target site in vivo 2 days or more after the administration. Combinations of the above-mentioned are also possible. For example, in some embodiments, a biomaterial preparation for use in accordance with the present disclosure is characterized in that, when assessed in vivo by administering to a target site (e.g., at and/or near a lymph node resection site) in a test subject (e.g., as described herein), 30%-80% or 40%-70% of such a relevant biomaterial preparation in a polymer network state remains at the target site in vivo 2 days or more after the administration.

In some embodiments, a biomaterial preparation for use in accordance with the present disclosure is characterized in that, when assessed in vivo by administering to a target site (e.g., at and/or near a lymph node resection site) in a test subject (e.g., as described herein), at least 10% or more, including, e.g., at least 20%, at least 30%, at least 40%, at least 50%, at least 60%, at least 70%, at least 80%, at least 90%, or more, of such a relevant biomaterial preparation in a polymer network state remains at the target site in vivo 3 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a relevant biomaterial preparation in a polymer network state remains at a target site in vivo 3 days or more after the administration. Combinations of the above-mentioned are also possible. For example, in some embodiments, a relevant biomaterial preparation is characterized in that, when assessed in vivo by administering to a target site (e.g., at and/or near a lymph node resection site) in a test subject (e.g., as described herein), 30%-80% or 40%-70% of such a relevant biomaterial preparation in a polymer network state remains at the target site in vivo 3 days or more after the administration.

In some embodiments, a biomaterial preparation for use in accordance with the present disclosure is characterized in that, when assessed in vivo by administering to a target site (e.g., at and/or near a lymph node resection site) in a test subject (e.g., as described herein), at least 10% or more, including, e.g., at least 20%, at least 30%, at least 40%, at least 50%, at least 60%, at least 70%, at least 80%, at least 90%, or more, of such a relevant biomaterial preparation in a polymer network state remains at the target site in vivo 5 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a relevant biomaterial preparation in a polymer network state remains at a target site in vivo 5 days or more after the administration. Combinations of the above-mentioned are also possible. For example, in some embodiments, a relevant biomaterial preparation is characterized in that, when assessed in vivo by administering to a target site (e.g., at and/or near a lymph node resection site) in a test subject (e.g., as described herein), 30%-80% or 40%-70% of such a relevant biomaterial preparation in a polymer network state remains at the target site in vivo 5 days or more after the administration.

In some embodiments, a biomaterial preparation for use in accordance with the present disclosure is characterized in that, when assessed in vivo by administering to a target site (e.g., at and/or near a lymph node resection site) in a test subject (e.g., as described herein), at least 10% or more, including, e.g., at least 20%, at least 30%, at least 40%, at least 50%, at least 60%, at least 70%, at least 80%, at least 90%, or more, of such a relevant biomaterial preparation in a polymer network state remains at the target site in vivo 7 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a relevant biomaterial preparation in a polymer network state remains at a target site in vivo 7 days or more after the administration. Combinations of the above-mentioned are also possible. For example, in some embodiments, a relevant biomaterial preparation is characterized in that, when assessed in vivo by administering to a target site (e.g., at and/or near a lymph node resection site) in a test subject (e.g., as described herein), 30%-80% or 40%-70% of such a relevant biomaterial preparation in a polymer network state remains at the target site in vivo 7 days or more after the administration.

In some embodiments, a biomaterial preparation for use in accordance with the present disclosure is characterized in that, when assessed in vivo by administering to a target site (e.g., at and/or near a lymph node resection site) in a test subject (e.g., as described herein), at least 10% or more, including, e.g., at least 20%, at least 30%, at least 40%, at least 50%, at least 60%, at least 70%, at least 80%, at least 90%, or more, of such a relevant biomaterial preparation in a polymer network state remains at the target site in vivo 14 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a relevant biomaterial preparation in a polymer network state remains at a target site in vivo 14 days or more after the administration. Combinations of the above-mentioned are also possible. For example, in some embodiments, a relevant biomaterial preparation is characterized in that, when assessed in vivo by administering to a target site (e.g., at and/or near a lymph node resection site) in a test subject (e.g., as described herein), 30%-80% or 40%-70% of such a relevant biomaterial preparation in a polymer network state remains at the target site in vivo 14 days or more after the administration.

In some embodiments, a biomaterial preparation for use in accordance with the present disclosure is characterized in that, when assessed in vivo by administering to a target site (e.g., at and/or near a lymph node resection site) in a test subject (e.g., as described herein), no more than 10% or less, including, e.g., no more than 9%, no more than 8%, no more than 7%, no more than 6%, no more than 5%, no more than 4%, no more than 3%, no more than 2%, no more than 1% or less, of such a relevant biomaterial preparation in a polymer network state remains at the target site in vivo 10 days or more after the administration.

In certain embodiments, compositions described herein comprise a biomaterial preparation that forms a matrix or depot and a immunomodulatory payload that is within the biomaterial preparation. In certain embodiments, a immunomodulatory payload is released from a biomaterial preparation after administration at a target site (e.g., at and/or near a lymph node resection site) by diffusion. For example, in certain embodiments, a polymer network state of a biomaterial preparation may be characterized in that, when tested in vitro by placing a composition comprising a biomaterial and an immunomodulatory payload in PBS (pH 7.4), less than 100% (including, e.g., less than 95%, less than 90%, less than 85%, less than 80%, less than 70%, less than 50%, or lower) of the immunomodulatory payload is released within 3 hours from the biomaterial preparation.

In certain embodiments, a polymer network state of a biomaterial preparation is characterized in that, when tested in vitro by placing a composition comprising a biomaterial and an immunomodulatory payload in PBS (pH 7.4), at least 30% (including, e.g., at least 35%, at least 40%, at least 45%, at least 50%, at least 55%, at least 60%, at least 65%, or more) of the immunomodulatory payload is released within 12 hours from the biomaterial preparation.

In certain embodiments, a polymer network state of a biomaterial preparation is characterized in that, when tested in vivo by administering a composition comprising a biomaterial and a immunomodulatory payload at a mammary fat pad of a mouse subject, less than or equal to 60% (including, e.g., less than or equal to 50%, less than or equal to 40%, etc.) of the immunomodulatory payload is released in vivo 8 hours after the administration.

In some embodiments, a composition provided herein is characterized in that a test animal group with spontaneous metastases having, at a target site (e.g., at and/or near a lymph node resection site), such a composition has a higher percent survival than that of a comparable test animal group having, at a target site (e.g., at and/or near a lymph node resection site), a biomaterial preparation without an immunomodulatory payload, as assessed at 2 months after the administration. In some such embodiments, an increase in percent survival as observed in a test animal group with spontaneous metastases having, at a target site (e.g., at and/or near a lymph node resection site), an immunomodulatory composition is at least 30% or more, including, at least 40%, at least 50%, at least 60%, at least 70%, at least 80%, at least 90%, or more, as compared to that of a comparable test animal group having, at a target site (e.g., at and/or near a lymph node resection site), a biomaterial preparation without an immunomodulatory payload, as assessed at 2 months after the administration.

In some embodiments, a composition provided herein is characterized in that a test animal group with spontaneous metastases having, at a target site (e.g., at and/or near a lymph node resection site), such a composition has a higher percent survival than that of a comparable test animal group having, at a target site (e.g., at and/or near a lymph node resection site), a biomaterial preparation without an immunomodulatory payload, as assessed at 3 months after the administration. In some such embodiments, an increase in percent survival as observed in a test animal group with spontaneous metastases having, a target site (e.g., at and/or near a lymph node resection site), an immunomodulatory composition is at least 10% or more, including, at least 20%, at least 30%, at least 40%, at least 50%, at least 60%, at least 70%, at least 80%, at least 90%, or more, as compared to that of a comparable test animal group having, at a target site (e.g., at and/or near a lymph node resection site), a biomaterial preparation without a immunomodulatory payload, as assessed at 3 months after the administration.

In certain embodiments, biomaterial preparations described herein may form polymer networks with or without the addition of a cross-linking agent. In certain embodiments, a polymer network is crosslinked. Polymer networks (e.g., hydrogels) can be crosslinked using any methods known in the art, e.g., chemical crosslinking methods (e.g., by using a small-molecule cross-linker, which can be derived from a natural source or synthesized), polyelectrolyte crosslinking (e.g., mixing a polymer with a second polymer comprising an opposite charge), thermal-induced crosslinking, photo-induced crosslinking (e.g., using vinyl sulfone, methacrylate, acrylic acid), pH-induced crosslinking, and enzyme-catalyzed crosslinking. In some embodiments, one or more cross-linking methods described in Parhi, Adv Pharm Bull., Review 7(4): 515-530 (2017); which is incorporated herein by reference for the purposes described herein, can be used in forming a polymer network (e.g., a hydrogel).

B. Exemplary Payloads

As already noted herein, in some embodiments, biomaterial preparations (e.g., polymer preparations as described herein, including for example polymer combination preparations) may be administered without an additional payload; in some embodiments, such preparations may themselves have certain immunomodulatory properties. Alternatively or additionally, in some embodiments, biomaterial preparations (e.g., polymer preparations as described herein, including for example polymer combination preparations) may comprise and/or otherwise be administered in combination with one or more therapeutic agents and/or payloads (e.g., immunomodulatory payloads, e.g., immunomodulatory agents). That is, in some embodiments, an immunomodulatory composition may comprise or consist of a biomaterial (e.g., a polymer) and a payload.

In some embodiments, an immunomodulatory composition as described herein includes one or more payloads. In some embodiments, a payload may be or comprise a biologically active agent (e.g., whose administration to a site as described herein has a biological (e.g., physiological) impact on the site); in some embodiments, a payload may be or comprise a detectable agent (e.g., a detectable moiety).

In some embodiments, a payload is an immunomodulatory agent.

In some embodiments, a payload is not a toxic (e.g., cytotoxic or cytostatic, or other antiproliferative) agent, e.g., is not a traditional chemotherapeutic agent that acts simply by killing cancer cells, but does not promote a clinically relevant extent of immunogenic cell death (for example, see: Vacchelli et al., “Trial watch: Chemotherapy with immunogenic cell death inducers”, Oncoimmunology, Mar. 1, 2013; Kepp et al., “Consensus guidelines for the detection of immunogenic cell death” Oncoimmunology, Dec. 13, 2014; Bloy et al., “Immunogenic stress and death of cancer cells: Contribution of antigenicity vs adjuvanticity to immunosurveillance” Immunology Reviews, November 2017; Michaud et al., “Autophagy-dependent anticancer immune responses induced by chemotherapeutic agents in mice”, Science, Dec. 16, 2011; Galluzzi et al., “Molecular mechanisms of cell death: recommendations of the Nomenclature Committee on Cell Death 2018”, Cell Death Differentiation, March 2018; Galluzzi et al., “Immunogenic cell death in cancer and infectious disease”, Nature Reviews Immunology, October 2016; Galluzzi et al., “Immunostimulation with chemotherapy in the era of immune checkpoint inhibitors”, Nature Reviews Clinical Oncology, Aug. 5, 2020; the contents of each of which are incorporated herein by reference for purposes described herein). In some embodiments, whether such a chemotherapeutic agent can promote a clinically relevant extent of immunogenic cell death can be determined, for example, by assessing for relative therapeutic benefit of the chemotherapeutic agent following treatment of the same tumor model in immunocompromised versus healthy mice. Examples of a traditional chemotherapeutic agent can be found among any of a variety of classes of anti-cancer agents including, but not limited to, alkylating agents, anti-metabolites, topoisomerase inhibitors, and/or mitotic inhibitors. In some embodiments, an immunomodulatory composition as described herein is substantially free of any traditional chemotherapeutic agent. In some embodiments, an immunomodulatory composition is substantially free of a cytotoxic or cytostatic agent (or other antiproliferative agent).

In some embodiments, a payload may be dispersed within a biomaterial preparation (e.g., a polymer preparation as described herein, including for example a polymer combination preparation). In some embodiments, the present disclosure, among other things, provides and/or utilizes a composition comprising a polymer preparation and/or one or more payloads, wherein at least some of the payload(s) is dispersed within the polymer preparation.

In some embodiments, a payload that may be included in a biomaterial preparation may be or comprise a therapeutic agent for treatment and/or prophylaxis of a disease, disorder, or condition. In some embodiments, a therapeutic agent included in a biomaterial preparation may be or comprise an agent for immunomodulation, wound healing, cancer therapy, and/or analgesia. In some embodiments, a therapeutic agent included in a biomaterial preparation may be useful for treatment of cancer. In some embodiments, a payload that may be included in a biomaterial preparation is or comprises a chemotherapeutic agent, for example, in some embodiments a chemotherapeutic agent that induces immunogenic cell death. As will be recognized by one of ordinary skill in the art, such a chemotherapeutic agent suitable for use in accordance with the present disclosure may be a synthetic or natural compound; a single molecule or a complex of different molecules. In some embodiments, suitable chemotherapeutic agents that induces immunogenic cell death can belong to any of various classes of compounds including, but not limited to, small molecules, peptides, saccharides, steroids, antibodies, fusion proteins, nucleic acid agents (e.g., but not limited to antisense polynucleotides, ribozymes, and small interfering RNAs), peptidomimetics, and the like.

In some embodiments, a payload that may be included in a biomaterial preparation preparation) is or comprises one or more nucleic acid agents. Such a nucleic acid agent may have enzymatic activity (e.g., ribozyme activity), gene expression inhibitory activity (e.g., as an antisense or interfering RNA agent, etc.), polypeptide-encoding activity, immunomodulatory activity, and/or other activities. In some embodiments, a nucleic acid agent that may be included in a biomaterial preparation may itself act to modulate one or more aspects of an immune response, or may encode a modulator of one or more aspects of an immune response.

In some embodiments, a payload that may be included in a biomaterial preparation is or comprises a photosensitizer used in photodynamic therapy (PDT). In PDT, local or systemic administration of a photosensitizer to a patient is followed by irradiation with light that is absorbed by the photosensitizer in the tissue or organ to be treated. Light absorption by the photosensitizer generates reactive species (e.g., radicals) that are detrimental to cells. For maximal efficacy, a photosensitizer not only has to be in a form suitable for administration, but also in a form that can readily undergo cellular internalization at the target site, preferably with some degree of selectivity over normal tissues.

In some embodiments, a payload is or comprises a radiosensitizer. A radiosensitizer is typically a molecule, compound or agent that makes target cells more sensitive to radiation therapy. Administration of an immunomodulatory composition (e.g., that is or comprises a biomaterial preparation comprising a radiosensitizer to a patient receiving radiation therapy may concentration function of the radiosensitizer on target cells and thereby enhance the effects of radiation therapy.

In some embodiments, a payload is or comprises a radioisotope. Examples of suitable radioisotopes include any α-, β-, or γ-emitter, which, when localized at a target site, results in cell destruction, including, e.g., but not limited to Examples of such radioisotopes include, but are not limited to, iodine-131, iodine-125, bismuth-212, bismuth-213, astatine-211, rhenium-186, rhenium-188, phosphorus-32, yttrium-90, samarium-153, and lutetium-177.

In some embodiments, a payload is or comprises a prodrug activating enzyme, e.g., for a directed enzyme prodrug therapy approach. For example, in some embodiments, a biomaterial preparation comprising a prodrug activating enzyme and a prodrug can be administered to a subject, wherein the biomaterial preparation forms in situ at a target site and the prodrug activating enzyme included therein converts the prodrug delivered to/around the target site into an active drug. The prodrug can be converted to an active drug in one step (by the prodrug activating enzyme) or in more than one step.

In some embodiments, a payload is or comprises an anti-angiogenic agent. Anti-angiogenic agents suitable for use in accordance with the present disclosure may include any molecule, compound or factor that blocks, inhibits, slows down or reduce the process of angiogenesis, or the process by which new blood vessels form by developing from pre-existing vessels. Such a molecule, compound or factor can block angiogenesis by blocking, inhibiting, slowing down or reducing any of the steps involved in angiogenesis, including the steps of (1) dissolution of the membrane of the originating vessel, (2) migration and proliferation of the endothelial cells, and (3) formation of new vascular tube by the migrating cells. Examples of anti-angiogenic agents include, but are not limited to, bevacizumab (Avastin®), celecoxib (Celebrex®), endostatin, anti-VEGF antibody, interferon-α, squalamine, cisplatin, combretastatin A-4, and Neovastat.

In some embodiments, a payload is or comprises an immunomodulatory payload. In some embodiments, an immunomodulatory payload is included in a biomaterial preparation as monotherapy. In some embodiments, an immunomodulatory payload is or comprises a modulator of inflammation. As will be understood by appreciated by one of skilled in the art, inflammation may be immunostimulatory or immunosuppressive depending on the biological context. Accordingly, in some embodiments, an immunomodulatory payload is or comprises a modulator of immunostimulatory inflammation. In some embodiments, an immunomodulatory payload is or comprises a modulator of immunosuppressive inflammation. In some embodiments, an immunomodulatory payload is or comprises a modulator of innate immunity and/or adaptive immunity. In some such embodiments, a modulator of innate immunity and/or adaptive immunity is or comprises an agonist of innate immunity and/or adaptive immunity.

In some embodiments, an immunomodulatory payload is or comprises a modulator of granulocytes. Granulocytes are a category of white blood cells in an innate immune system characterized by the presence of granules in their cytoplasm. Granulocytes may also be referred to as polymorphonuclear leukocytes or polymorphonuclear neutrophils (PMN, PML, or PMNL) because of the varying shapes of the nucleus, which is usually lobed into three segments. This distinguishes them from mononuclear agranulocytes. Examples of granulocytes include but are not limited to neutrophils, eosinophils, basophils, and/or mast cells.

In some embodiments, an immunomodulatory payload is or comprises a modulator of agranulocytes. As appreciated by one of skilled in the art, agranulocytes, also known as nongranulocytes or mononuclear leukocytes, are characterized by the absence of granules in their cytoplasm, which distinguishes them from granulocytes. Examples of agranulocytes include but are not limited to lymphocytes, monocytes, and/or macrophages. Lymphocytes, as will be understood by one of skilled in the art, typically include but are not limited to B cells, T cells, natural killer T cells, and/or natural killer (NK) cells.

In some embodiments, an immunomodulatory payload is or comprises a modulator of myeloid cells and/or lymphoid cells. In some embodiments, an immunomodulatory payload is or comprises a modulator of neutrophils, eosinophils, basophils, lymphocytes, and/or monocytes. In some embodiments, an immunomodulatory payload is or comprises a modulator of hematopoietic stem cells, common myeloid progenitors, megakaryocytes, thrombocytes, erythrocytes, mast cells, myeloblasts, basophils, neutrophils, eosinophils, monocytes, macrophages, dendritic cells, common lymphoid progenitors, natural killer cells, T lymphocytes, B lymphocytes, and/or plasma cells.

In some embodiments, an immunomodulatory payload is or comprises an immunomodulatory agent as described in International Patent Publication No. WO 2018/045058 (which includes, e.g., but not limited to examples of activators of innate immune response, activators of adaptive immune response, immunomodulatory cytokines, modulators of macrophage effector functions, etc.) and WO 2019/183216 (which includes, e.g., but not limited to inhibitors of immunosuppressive inflammation, e.g., mediated by a p38 mitogen-activated protein kinase (MAPK) pathway, etc.), the contents of each of which are incorporated herein by reference for purposes described herein. In some embodiments, an immunomodulatory payload is or comprises an activator of innate immune response, for example, in some embodiments, which may be or comprise a stimulator of interferon genes (STING) agonist, a Toll-like receptor (TLR) agonist, and/or an activator of innate immune response as described in International Patent Publication No. WO 2018/045058, the contents of which are incorporated herein by reference for purposes described herein. In some embodiments, an immunomodulatory payload is or comprises an inhibitor of immunosuppressive inflammation, for example, in some embodiments, which may be or comprise a COX-2 inhibitor or an inhibitor of immunosuppressive inflammation mediated by a p38 mitogen-activated protein kinase (MAPS) pathway, as described in International Patent Publication No. WO 2019/183216, the contents of which are incorporated herein by reference for purposes described herein.

In some embodiments, an immunomodulatory payload is or comprises a Toll-like receptor 7 and 8 (TLR7/8) agonist (e.g., ones described in the International Patent Publication No. WO 2018/045058). In some embodiments, an exemplary TLR7/8 agonist is or comprises resiquimod (R848) or a variant thereof.

In some embodiments, an immunomodulatory payload is or comprises a immunomodulatory cytokine (e.g., ones described in the International Patent Publication No. WO 2018/045058). In some embodiments, an exemplary immunomodulatory cytokine is or comprises interleukin-2 (IL-2), interleukin-12 (IL-12), or a variant thereof.

In some embodiments, an immunomodulatory payload is or comprises a non-steroidal anti-inflammatory drug (NSAID) (e.g., ones described in the International Patent Publication No. WO 2019/183216). In some embodiments, a NSAID is or comprises ketorolac. Ketorolac has been conventionally used for short-term pain management and, therefore, is typically not prescribed for longer than five days owing to toxicity. Systemic exposure of ketorolac can lead to renal and cardiac toxicity as well as bleeding in the gastrointestinal tract. In some embodiments, the present disclosure appreciates that local retention of ketorolac may be desirable. For example, in some embodiments, ketorolac for use in the present disclosure is released from a biomaterial preparation (e.g., from a polymer preparation such as a polymer combination preparation as described herein) over a period of at least 3 hours or longer, e.g., at least 6 hours, at least 12 hours, at least 1 day, at least 2 days, at least 3 days, at least 4 days, at least 5 days, at least 6 days, at least 7 days, at least 8 days, at least 9 days, at least 10 days, or longer such that the immune system is modulated (e.g., immunosuppressive inflammation induced by tumor resection surgery is inhibited or reduced). Ketorolac may be administered as a racemic mixture or as an individual enantiomer, e.g., the S-enantiomer. In some embodiments, a NSAID comprises lornoxicam. In some embodiments, a NSAID comprises meclofenamate sodium.

In some embodiments, an immunomodulatory payload is or comprises a resolvin (e.g., ones as described in the International Patent Publication No. WO 2019/183216). In some embodiments, an exemplary resolving is or comprises RvD2. RvD2 is a resolvin that acts as a specialized pro-resolving mediator (SPM) involved in a coordinated resolution program that can prevent excessive inflammation and/or resolve acute inflammation.

In some embodiments, an immunomodulatory payload is or comprises a hematopoietic stem cell mobilizer and/or a CXCR4 receptor antagonist. In some embodiments, a hematopoietic stem cell mobilizer and/or a CXCR4 receptor antagonist is plerixafor.

In some embodiments, an immunomodulatory payload is or comprises a NOD1 and/or NOD2 agonist (e.g., as described in the International Patent Publication No. WO 2018/045058). In some embodiments, an exemplary NOD1 and/or NOD2 agonist may be or comprises L-Ala-γ-D-Glu-mDAP (TriDAP). Tri-DAP is typically present in the peptidoglycan (PGN) of Gram-negative bacilli and certain Gram-positive bacteria. In some embodiments, Tri-DAP is recognized by the intracellular sensor NOD1, which induces a signaling cascade leading to NF-κB activation and/or production of inflammatory cytokines. In some embodiments, an exemplary NOD1 and/or NOD2 agonist may be or comprises MurNAc-L-Ala-γ-D-Glu-mDAP (M-TriDAP). Similar to TriDAP, M-TriDAP is a peptidoglycan (PGN) degradation product found mostly in Gram-negative bacteria. M-TriDAP is typically recognized by the intracellular sensor NOD1 (CARD4) and to a lesser extent NOD2 (CARD15). Recognition of M-TriDAP by NOD 1/NOD2 induces a signaling cascade involving the serine/threonine RIP2 (RICK, CARDIAK) kinase, which interacts with IKK leading to the activation of NF-κB and production of inflammatory cytokines such as TNF-α and IL-6. In some embodiments, M-TriDAP induces the activation of NF-κB at similar levels to Tri-DAP.

In some embodiments, an immunomodulatory payload is or comprises a modulator of an immune cell effector function, survival, and/or recruitment. In some embodiments, an immunomodulatory payload is or comprises a modulator of monocyte effector function, survival, and/or recruitment. In some embodiments, an immunomodulatory payload is or comprises a modulator of a macrophage effector function, survival, and/or recruitment. In some embodiments, an immunomodulatory payload is or comprises a modulator of myeloid derived suppressor cell effector function, survival, and/or recruitment. In some embodiments, an immunomodulatory payload is or comprises a modulator of neutrophil function, survival, and/or recruitment. In some embodiments, an immunomodulatory payload is or comprises a modulator of natural killer cell effector function, survival, and/or recruitment. Examples of such modulators of immune cell effector function, survival, and/or recruitment may include, but are not limited to adenosine A2A receptor (A2AR) inhibitors, chemokines (e.g., CCL1, CCL2, CCL3, CCL4, CCL5, CCL17, CCL19, CCL21, CCL22, CXCL9, CXCL10, CXCL11, CXCL13, CXCL16, and/or CX3CL1, etc.), angiopoietin 2 (ANG2) inhibitors, arginase-1 (ARG1) inhibitors, colony-stimulating factor 1 (CSF1) inhibitors, granulocyte-macrophage-colony-stimulating factor (GM-CSF) inhibitors, colony-stimulating factor 1 receptor (CSF1R) inhibitors, ectonucleoside triphosphate diphosphohydrolase (ENTPD1, also known as CD39) inhibitors, tumor necrosis factor receptor superfamily member 5 (CD40) agonists, OX40 agonists, 4-1BB agonists, CD160 agonists, DNAM agonists, NKG2D agonists, NKG2A inhibitors, TIGIT inhibitors, LILRB1 inhibitors, LILRB2 inhibitors, leukocyte surface antigen CD47 (CD47) inhibitors, signal regulatory protein alpha (SIRPα) inhibitors, 5′-nucleotidase (NT5E, also known as CD73) inhibitors, prostaglandin-endoperoxide synthase 2 (PTGS2, also known as cyclooxygenase-2 (COX-2)) inhibitors, prostaglandin E2 (PGE2) inhibitors, PGE2 receptor 2 (EP2) inhibitors, PGE2 receptor 4 (EP4) inhibitors, inducible nitric oxide synthase (iNOS) inhibitors, fibroblast growth factor 1 (FGF) inhibitors, indoleamine 2,3-dioxygenase (IDO) inhibitors, Class II HDAC (e.g., HDAC4, HDAC5, HDAC6, HDAC7, HDAC9, and HDAC10) inhibitors, Ig-Like Transcript 2 (ILT2) inhibitors, S100A8/A9 inhibitors, RAGE inhibitors, interleukin-8 (IL-8, also known as CXCL8) inhibitors, C-X-C chemokine receptor type 1 (CXCR-1) inhibitors, C-X-C chemokine receptor type 2 (CXCR-2) inhibitors, interleukin-10 (IL-10) inhibitors, interleukin-2 (and variants thereof), interleukin-12 (IL-12) (including subunit alpha (IL-12a) and subunit beta (IL-12b)) (and variants thereof), interleukin-15 (and variants thereof), interleukin-18 (and variants thereof), Leukotriene B4 (LTB4) inhibitors, resolvin family (e.g., RvD1, RvD2, RvD3, RvD4, RvD5, RvD6, 17R-RvD1, 17R-RvD2, 17R-RvD3, 17R-RvD4, 17R-RvD5, 17RRvD6, RvE1, 18S-RvE1, RvE2, RvE3, RvT1, RvT2, RvT3, RvT4, RvD1ₙ-3, RvD2ₙ-3, and/or RvD5ₙ-3) specialized pro-resolving mediators (SPMs), lipoxin family (e.g., LxA4, LxB4, 15-epi-LxA4, and/or 15-epiLxB4) SPMs, protectin/neuroprotection (e.g., DHA-derived protectins/neuroprotectins and/or n-3 DPA-derived protectins/neuroprotectins) SPMs, maresins (e.g., DHA-derived maresins and/or n-3DPA-derived maresins) SPMs, phosphoinositide 3 kinase gamma (PI3Kγ) inhibitors, transforming growth factor beta (TGF-β) inhibitors, transforming growth factor beta receptors (TGF-βR family, e.g., ALK1, ALK2, ALK3, ALK4, TGF-βR1, ALK6, ALK7, TGF-βR2, TGF-βR3, BMPR2, ACVR2A, ACVR2B, and/or AMHR2), vascular endothelial growth factor family (VEGF, e.g., VEGF-A, VEGF-B, VEGF-C, and/or VEGF-D) inhibitors, vascular endothelial growth factor receptor family (VEGFR, e.g., VEGFR-1, VEGFR-2, and/or VEGFR-3) inhibitors, JAK/STAT inhibitors, angiotensin II receptor inhibitors (e.g., valsartan), and/or combinations thereof.

Those skilled in the art will appreciate that the human immune system is complex, and rigid classification of a particular agent as one category of immunomodulatory agent (e.g., as an agonist of innate immunity versus of adaptive immunity and/or as a modulator of macrophage effector function, of granulocytes, of myeloid cells and/or lymphoid cells, etc.) is not always useful, necessary, or sometimes even possible. Those skilled in the art, based on descriptions herein, will understand in context the metes and bounds of relevant agents useful in embodiments as described herein. For example, in some embodiments, certain immunomodulatory agents that may be useful as activators of adaptive immune response in one context may be also effective to modulate survival, recruitment, and/or effector function of one or more immune cell types, including, e.g., macrophages, monocytes, myeloid-derived suppressor cells, neutrophils, and/or natural killer cells.

In some embodiments, an immunomodulatory payload is released from an immunomodulatory composition and is taken up by immune cells. In some embodiments, immune cells that take up the immunomodulatory payload exhibit at least one of the following biological activities: expressing an immunomodulatory polypeptide in response to an immunomodulatory payload, exhibiting an increased expression of a type 1 interferon in response to innate immune stimulation induced by an immunomodulatory payload, and/or exhibiting a change in level and/or activity of an immunomodulatory polypeptide.

In some embodiments, an immunomodulatory payload is a polynucleotide agent. In some embodiments, a polynucleotide agent is a coding polynucleotide that can be translated into a polypeptide. In some embodiments, a biomaterial preparation included in an immunomodulatory composition is characterized in that the polynucleotide agent is released from the biomaterial preparation and is taken up by local cells so that at least a subset of local immune cells express the immunomodulatory polypeptide encoded by the polynucleotide agent. In some embodiments, a biomaterial preparation included in an immunomodulatory composition is characterized in that at least a subset of local immune cells have an increased expression of a type 1 interferon in response to innate immune stimulation induced by the polynucleotide agent. In some embodiments, a biomaterial preparation included in an immunomodulatory composition is characterized in that, at least a subset of local immune cells have a change in level and/or activity of an immunomodulatory polypeptide in response to the polynucleotide agent.

In some embodiments, a target cell may comprise myeloid cells and/or plasmacytoid dendritic cells. In some embodiments, a target cell may comprise non-immune cells, such as fibroblasts and/or endothelial cells.

### II. Pharmaceutical Compositions

In some embodiments, a biomaterial preparation for use in accordance with the present disclosure can be formulated in accordance with available technologies as a pharmaceutical composition (e.g., an immunomodulatory composition) for administration to a subject in need thereof (e.g., as described herein). In some embodiments, such a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, M D, 2006; the contents of which are incorporated herein by reference for purposes described herein) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, lactose, trehalose, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In some embodiments, a pharmaceutical composition can be sterile. A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a pharmaceutical composition for injection may typically comprise sterile isotonic aqueous buffer. Where necessary, a pharmaceutical composition may also include a local anesthetic to ease pain at a site of injection. In some embodiments, components of a pharmaceutical composition (e.g., as described herein) are supplied separately or mixed together in a single-use form, for example, as a dry lyophilized powder or water free concentrate in a hermetically sealed container such as an ampule or sachet or in a sterile syringe indicating the quantity of a composition comprising a biomaterial preparation (e.g., ones described herein). Where a pharmaceutical composition is to be administered by injection, in some embodiments, a dry lyophilized powder composition comprising a biomaterial preparation (e.g., ones described herein) can be reconstituted with an aqueous buffered solution and then injected to a target site in a subject in need thereof. In some embodiments, a liquid composition comprising a biomaterial preparation (e.g., ones described herein) can be provided in a syringe for administration by injection and/or by a robotic surgical system (e.g., a da Vinci System).

In some embodiments, a liquid composition can be provided in a syringe for administration with or without a needle, cannula, or trocar.

In some embodiments, a liquid composition can be administered by spraying.

In some embodiments, administration of a liquid composition can be gas assisted for use in minimally invasive surgery.

In some embodiments, administration of a liquid composition can be achieved by using a multi-barrel syringe, with each barrel containing a separate polymer component of a biomaterial preparation, the multiple of which are combined upon depression of the shared plunger.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of pharmaceutical compositions described herein may be prepared in accordance with established pharmacology practices. For example, in some embodiments, such preparatory methods may include step of bringing components of a biomaterial preparation comprising an immunomodulatory payload into association with a diluent or another excipient and/or one or more other accessory ingredients and then, if necessary and/or desirable, shaping and/or packaging the product into a desired single-use unit or multi-use units. Alternatively, such preparatory methods may also include a step of pre-forming a biomaterial preparation comprising an immunomodulatory payload from described herein, prior to shaping and/or packaging the product into a desired single-use units or multi-use units.

In some embodiments, a pharmaceutical composition may be prepared, packaged, and/or sold in bulk, as a single-use unit, and/or as a plurality of single-use units. As used herein, a “single-use unit” is a discrete amount of a pharmaceutical composition described herein. For example, a single-use unit of a pharmaceutical composition comprises a predetermined amount of a composition and/or a biomaterial preparation comprising an immunomodulatory payload described herein, which in some embodiments can be or comprise a pre-formed polymer network of a biomaterial preparation comprising an immunomodulatory payload (e.g., ones described herein), or in some embodiments can be or comprise a liquid or a colloidal mixture of individual components of a biomaterial preparation (e.g., ones described herein).

The relative amount of individual components of a biomaterial preparation for use in accordance with the present disclosure (e.g., as a pre-formed polymer network biomaterial or as precursor component(s) of such a polymer network biomaterial) and, optionally, any additional agents in pharmaceutical compositions described herein, e.g., a pharmaceutically acceptable excipient and/or any additional ingredients, can vary, depending upon, e.g., desired material properties of a polymer biomaterial, size of target site, injection volume, physical and medical condition of a subject to be treated, and/or types of cancer, and may also further depend upon the route by which such a pharmaceutical composition is to be administered.

In some embodiments, an immunomodulatory composition (e.g., comprising a biomaterial, such as a polymer biomaterial, alone or in combination with a payload and, optionally, one or more other agents) in an amount effective to achieve (e.g., to have been established to achieve, when administered to a relevant population) a desired therapeutic effect (e.g., but not limited to inducing anti-tumor immunity in at least one or more aspects, e.g., inducing innate immunity).

In some embodiments, an effective amount is an amount useful for treatment of cancer. In some embodiments, an effective amount is an amount effective to inhibit or reduce risk or incidence of tumor recurrence and/or metastasis. In certain embodiments, an effective amount is a therapeutically effective amount. In certain embodiments, an effective amount is a prophylactically effective amount.

In certain embodiments, a pharmaceutical composition consists essentially of or consists of a biomaterial preparation comprising an immunomodulatory payload; to the extent that such a composition may include one or more material(s)/agents other than the biomaterial preparation comprising an immunomodulatory payload, such other material(s)/agent(s) do not, individually, or together, materially alter relevant immunomodulatory characteristic(s), of the biomaterial preparation comprising an immunomodulatory payload.

In certain embodiments, pharmaceutical compositions utilized in accordance with the present disclosure do not include cells. In certain embodiments, pharmaceutical compositions do not include adoptively transferred cells. In certain embodiments, pharmaceutical compositions do not include T cells. In certain embodiments, pharmaceutical compositions do not include tumor antigens. In certain embodiments, pharmaceutical compositions do not include tumor antigens loaded ex vivo.

In certain embodiments, a pharmaceutical composition is in liquid form (e.g., a solution or a colloid). In certain embodiments, a pharmaceutical composition is in a solid form (e.g., a gel form). In certain embodiments, the transition from a liquid form to a solid form may occur outside a subject's body upon sufficient crosslinking such that the resulting material has a storage modulus consistent with a solid form that allows it to be physically manipulated and implanted in a surgical procedure. Accordingly, in some embodiments, a solid form may be amenable for carrying out an intended use of the present disclosure (e.g., surgical implantation). In certain embodiments, the transition from a liquid form to a solid form may occur upon thermal crosslinking in situ (e.g., inside a body of a subject) such that the resulting material has a storage modulus consistent with a solid form. In certain embodiments, a pharmaceutical composition is a suspension.

### III. Methods of Treatment

Technologies provided herein are useful for treatment of cancer. In some embodiments, technologies provided herein are useful to delay the onset of, slow the progression of, and/or ameliorate one or more symptoms of cancer, particularly metastatic, disseminated, or post-tumor resection cancers in a subject. In some embodiments, technologies provided herein are useful to reduce or inhibit primary tumor growth or regrowth (e.g., after surgical resection). In some embodiments, technologies provided herein are useful to reduce or inhibit incidence of tumor recurrence and/or metastasis in a subject. In some embodiments, technologies provided herein are useful to reduce or inhibit incidence of secondary tumor occurrence in a subject. In some embodiments, technologies provided herein are useful to reduce or inhibit incidence of tumor dissemination in a subject. In some embodiments, technologies provided herein are useful for inducing anti-tumor immunity in a subject. In some embodiments, technologies provided herein are useful to reduce activation of metastasis otherwise observed upon or otherwise in association with lymph node resection.

In some embodiments, provided technologies are particularly useful for application to subjects that have undergone or will undergo a tumor resection.

In some embodiments, provided technologies are particularly useful for application to subjects who have received or are receiving traditional chemotherapy.

In some embodiments, provided technologies are associated with reduced incidence and/or severity of certain adverse events (e.g., skin rashes, hepatitis, diarrhea, colitis, hypophysitis, thyroiditis, and adrenal insufficiency) that can be associated with systemic administration of immunotherapeutic agents. Among other things, provided technologies may reduce or eliminate exposure of non-tumor-specific immune cells to systemically-administered immunotherapeutic drug(s) and/or to high doses of such drug(s) that are often required in order for systemic administration to achieve sufficient concentration in the tumor and/or metastatic site to induce a desired response; among other things, provided technologies may achieve local immunomodulation (e.g., local agonism of innate and/or adaptive immunity), at a site of lymph node. Without wishing to be bound by any particular theory, it is proposed that local administration as described herein, can among other things, improve effectiveness of a particular payload agent (e.g., an immunomodulatory payload), for example by concentrating the immunomodulatory effect where it is needed.

Additionally or alternatively, in some embodiments, provided technologies that provide local immunomodulation (e.g., agonism of innate and/or adaptive immunity) prior to or following lymph node resection can, among other things, break local immune tolerance toward cancer and allow for development of systemic antitumor immunity, which can, for example, in some embodiments, lead to eradiation of disseminated disease.

In some embodiments, a composition as described herein is administered as a liquid; in some embodiments, a composition as described herein is administered as a gel.

In some embodiments, an administered composition as described herein does not comprise polymer particles and/or does not comprise payload encapsulated within or otherwise in a particle format.

In general, the present disclosure provides technologies whereby an immunomodulatory composition as described herein is administered to a lymph node resection site, typically in association with (e.g., as part of the same surgical procedure) such lymph node resection.

In some embodiments, the lymph node resection site is in the vicinity of a tumor (e.g., a primary tumor, an established metastasis, etc). In some embodiments, resected tissue comprises tumor cells; in some embodiments, resected tissue is substantially free of tumor cells. In some embodiments, a lymph node resection site is in immunological communication with a tumor site of the subject. In some embodiments, a resected lymph node is or comprises a sentinel lymph node.

In some embodiments, compositions as described herein are administered through a method comprising injection. In some embodiments, administration may be or comprise injection with a hypodermic needle. In some embodiments, injection is or comprises subcutaneous injection. In some embodiments, a subcutaneous injection comprises injection into the fat layer underneath the skin. In some embodiments, a subcutaneous injection comprises injection into an appropriate target site (e.g., at and/or near a lymph node resection site).

In some embodiments, injection is or comprises injection into a void space created through surgical intervention (e.g., resection surgery).

In some embodiments, a step of administering and/or delivering a biomaterial preparation (e.g., as described herein, e.g., comprising an extended release composition) comprising a non-particle immunomodulatory agent comprises administering one or more precursors of the biomaterial preparation, so that the biomaterial preparation composition forms in situ, creating an extended release composition. In some embodiments, such an in situ formation comprises converting from a precursor composition into an extended release composition. In some embodiments, such a precursor composition has lower viscosity and/or storage modulus than a formed extended release composition. In some embodiments, such an extended release composition is created by a process comprising the conversion of precursor components by gelation and/or crosslinking (e.g., gelation is in response to temperature-induced crosslinking, pH-induced crosslinking, ionic crosslinking etc).

In some embodiments, technologies provided herein comprise delivery of a biomaterial preparation to a target site. In some embodiments, a target site may be but is not limited to a site from which part or all of at least one lymph node has been resected. In some embodiments, a relevant lymph node may be or comprise: a draining lymph node, a sentinel lymph node, a proximal lymph node, a lymph node in immunological communication with a tumor (e.g., a primary tumor and/or in some embodiments, an established metastasis.

In some embodiments, administration occurs during or otherwise in association with lymph node resection which, in some embodiments, may be performed separately from (typically, but not always, before) tumor resection. Alternatively, in some embodiments, lymph node resection is performed concurrently with tumor resection.

In some embodiments, technologies provided herein may be particularly useful for treatment of a subject that has not undergone tumor resection; in other embodiments, may be particularly useful for treatment of a subject who is undergoing tumor resection and in yet other embodiments for treatment of a subject who has undergone resection of at least a portion of a tumor. In some embodiments, technologies provided herein may be particularly useful for treatment of a subject that is not a candidate for tumor removal, e.g., a subject that cannot undergo tumor resection (e.g., due to health constraints, technological constraints, religious constraints, etc.). In some embodiments, technologies provided herein may be particularly useful for treatment of a subject that has a cancer showing characteristics of metastatic potential (e.g., cancers with mutations in tumor protein p53 (TP53), cyclin-dependent kinase inhibitor 2A (CDKN2A), phosphatase and tensin homolog (PTEN), phosphatidylinositol-4,5-bisphosphate 3-kinase catalytic subunit alpha (PIK3CA), retinoblastoma (RB1), etc., see e.g., Fares et al., Molecular principles of metastasis: a hallmark of cancer revisited, Signal transduction and targeted therapy, Mar. 12, 2020).

In some embodiments, technologies provided herein comprise providing to a subject in need thereof, a composition as described herein, at a site near (e.g., in the vicinity of) a lymph node resection site. In some embodiments, “near” a resection site may be, for example, up to 10 cm away e.g., up to 1 cm, up to 2 cm, up to 3 cm, up to 4 cm, up to 5 cm, up to 6 cm, up to 7 cm, up to 8 cm, up to 9 cm, or up to 10 cm away from a location from which a lymph node (or portion thereof) was removed.

### IV. Therapeutic Uses

Technologies provided herein are useful for treatment of cancer. In some embodiments, technologies provided herein are useful to delay the onset of, slow the progression of, or ameliorate one or more symptoms of cancer. In some embodiments, technologies provided herein are useful to reduce or inhibit development, growth, and/or distribution of a lymph node tumor or metastasis. In some embodiments, technologies provided herein are useful to reduce or inhibit primary tumor growth or regrowth (after resection). In some embodiments, technologies provided herein are useful to reduce or inhibit incidence of tumor recurrence and/or metastasis. In some embodiments, technologies provided herein are useful for inducing anti-tumor immunity. In some embodiments, technologies provided herein are particularly useful when a subject is suffering from or susceptible to a recurrent or disseminated cancer. In certain embodiments, technologies provided herein are useful for inflammation and/or inflammatory disorders (e.g., autoimmune diseases and/or arthritis).

Accordingly, some aspects provided herein relate to methods of administering to a target site in a subject in need thereof an immunomodulatory composition (e.g., comprising a biomaterial preparation) described herein. In some embodiments, a subject receiving such a composition may be undergoing or may have undergone tumor removal (e.g., by surgical tumor resection). In some embodiments, a subject has not undergone tumor resection. In some embodiments, a subject receiving such a composition may have tumor relapse and/or metastasis. In some such embodiments, a method comprises intraoperative administration of a composition comprising a biomaterial preparation described herein at a target site (e.g., at and/or near a lymph node resection site); typically, a composition is administered during, or otherwise in association with, lymph node resection. In some embodiments, such an immunomodulatory composition utilized in methods of the present disclosure may be formulated as a pharmaceutical composition described herein.

In certain embodiments, technologies described herein comprise a method that comprises administering an immunomodulatory composition to a target site in a subject in need thereof during or after removal of tumor, for example, after removal of greater than or equal to 50% or higher, by weight, of the subject's tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by weight, of the subject's tumor. In certain embodiments, a method provided herein comprises administering an immunomodulatory composition to a target site in a subject in need thereof after removal of greater than or equal to 50% or higher, by volume, of the subject's tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by volume, of the subject's tumor. In some embodiments, a method provided herein comprises performing a tumor resection to remove a subject's tumor, prior to administration of an immunomodulatory composition.

In some embodiments, a composition described and/or utilized herein is administered to a target site (e.g., at and/or near a lymph node resection site) in a tumor resection subject immediately after the subject's lymph node has been removed, e.g., by surgical resection. In some embodiments, a composition described and/or utilized herein is intraoperatively administered to a target site in a lymph node resection subject. In some embodiments, a composition described and/or utilized herein is postoperatively administered one or more times to one or more target sites in a lymph node resection subject at one or more time points within 24 hours or less, including, e.g., within 18 hours, within 12 hours, within 6 hours, within 3 hours, within 2 hours, within 1 hour, within 30 mins, or less, after the subject's tumor has been removed by surgical lymph node resection. In some embodiments, a composition described and/or utilized herein is postoperatively administered one or more times to one or more target sites in a lymph node resection subject at one or more time points within 12 months or less from a surgical intervention, including e.g., within 11 months, within 10 months, within 9 months, within 8 months, within 7 months, within 6 months, within 5 months, within 4 months, within 3 months, within 2 months, or within 1 months of a surgical intervention. In some embodiments, a composition described and/or utilized herein is postoperatively administered one or more times to one or more target sites in a lymph node resection subject at one or more time points within 31 days, including e.g., within 30 days, within 29 days, within 28 days, within 27 days, within 26 days, within 25 days, within 24 days, within 23 days, within 22 days, within 21 days, within 20 days, within 19 days, within 18 days, within 17 days, within 16 days, within 15 days, within 14 days, within 13 days, within 12 days, within 11 days, within 10 days, within 9 days, within 8 days, within 7 days, within 6 days, within 5 days, within 4 days, within 3 days, within 2 days, or within 1 day of a surgical intervention.

In some embodiments, a target site for administration is or comprises a site at and/or near a lymph node at and/or near a tumor site (e.g., including a tumor resection site). In some embodiments, such a tumor site may be characterized by absence of gross residual tumor antigen. In some embodiments, such a tumor site may be characterized by a negative resection margin (i.e., no cancer cells seen microscopically at the resection margin, e.g., based on histological assessment of tissues surrounding the tumor resection site). In some embodiments, such a tumor site may be characterized by a positive resection margin (i.e., cancer cells are seen microscopically at the resection margin, e.g., based on histological assessment of tissues surrounding the tumor resection site). In some embodiments, such a tumor site may be characterized by presence of gross residual tumor antigen.

In some embodiments, a target site for administration is or comprises a site in immunological communication with a tumor site. In some embodiments, a target site for administration is or comprises a sentinel lymph node. In some embodiments, a target site for administration is or comprises a draining lymph node.

As will be understood by one of ordinary skill in the art, compositions that are useful in accordance with the present disclosure can be administered to a target site in subjects in need thereof using appropriate delivery approaches known in the art. For example, in some embodiments, provided technologies can be amenable for administration by injection. In some embodiments, provided technologies can be amenable for administration by minimally invasive surgery (MIS), e.g., robot-assisted MIS, robotic surgery, and/or laparoscopic surgery, which, for example, typically involve one or more small incisions. In some embodiments, provided technologies can be amenable for administration in the context of accessible and/or cutaneous excisions. In some embodiments, provided technologies can be amenable for administration (e.g., by injection) intraoperatively as part of minimally invasive procedure, e.g., minimally invasive surgery (MIS), e.g., robot-assisted MIS, robotic surgery, and/or laparoscopic surgery, and/or procedure that involves one or more accessible and/or cutaneous excisions. In some embodiments, provided technologies can be amenable for administration (e.g., by injection) involving a robotic surgical system (e.g., a da Vinci System), e.g., in some embodiments for minimally invasive administration. For example, in some embodiments, a composition that may be useful for injection and/or in the context of minimally invasive procedure, e.g., minimally invasive surgery (MIS), e.g., robot-assisted MIS, robotic surgery, and/or laparoscopic surgery and/or procedure that involves one or more accessible and/or cutaneous excisions, is liquid and a biomaterial preparation provided in such a composition is or comprises a polymer solution (e.g., a viscous polymer solution), which upon injection to a target site (e.g., at and/or near a lymph node resection site) in a subject, it transitions from a liquid solution state to a polymer network state (e.g., a hydrogel), which in some embodiments, such a transition is triggered by exposure to the body temperature of the subject. In some embodiments, a biomaterial preparation in a pre-formed polymer network biomaterial that is compressible without adversely impact its structural integrity can be injected, for example, by a minimally invasive procedure, e.g., minimally invasive surgery (MIS), e.g., robot-assisted MIS, robotic surgery, and/or laparoscopic surgery and/or procedure.

In some embodiments, technologies provided herein can be amenable for administration by implantation. For example, in some embodiments, a biomaterial preparation provided in a composition in accordance with the present disclosure is a pre-formed polymer network biomaterial. An exemplary polymer network biomaterial is or comprises a hydrogel. For example, in some embodiments, an immunomodulatory composition may be administered by surgical implantation to a target site (e.g., at and/or near a lymph node resection site). In some embodiments, an immunomodulatory composition may be administered by surgical implantation to target site (e.g., at and/or near a lymph node resection site) and affixed with a bioadhesive. In some embodiments, administration may be performed intraoperatively (i.e., immediately after tumor resection).

In some embodiments, the amount of immunomodulatory composition to achieve desirable therapeutic effect(s) such as, e.g., anti-tumor immunity, may vary from subject to subject, depending, for example, on gender, age, and general condition of a subject, type and/or severity of cancer, efficacy of an immunomodulatory composition, and the like.

In some embodiments, the present disclosure provides technologies such that administration of an immunomodulatory composition as described herein is sufficient to provide antitumor immunity and thus does not necessarily require administration of, e.g., a tumor antigen, and/or adoptive transfer of immune cells (e.g., T cells) to a subject in need thereof (e.g., as described herein). Accordingly, in some embodiments, technologies provided herein do not include administering a tumor antigen to a subject, e.g., within 1 month or less (including, e.g., within 3 weeks, within 2 weeks, within 1 week, within 5 days, within 3 days, within 1 day, within 12 hours, within 6 hours), after the subject has received a composition as described and/or utilized herein. In certain embodiments, technologies provided herein do not include adoptive transfer of immune cells (e.g., T cells) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In some embodiments, the present disclosure provides technologies such that administration of an immunomodulatory composition is particularly effective when administered as a co-therapy with e.g., a tumor antigen, and/or adoptive transfer of immune cells (e.g., T cells) to a subject in need thereof (e.g., as described herein). Accordingly, in some embodiments, technologies provided herein include administering a tumor antigen to a subject, e.g., within 1 month or less (including, e.g., within 3 weeks, within 2 weeks, within 1 week, within 5 days, within 3 days, within 1 day, within 12 hours, within 6 hours), after the subject has received a composition as described and/or utilized herein. In certain embodiments, technologies provided herein include adoptive transfer of immune cells (e.g., T cells) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In some embodiments, technologies provided herein are useful for treatment of cancer in a subject. In some embodiments, technologies provided herein are for use in treatment of a resectable tumor. In some embodiments, technologies provided herein are for use in treatment of a solid tumor (e.g., but not limited to a blastoma, a carcinoma, a germ cell tumor, and/or a sarcoma). In some embodiments, technologies provided herein are for use in treatment of lymphoma present in a spleen or a tissue outside of a lymphatic system, e.g., a thyroid or stomach.

In some embodiments, technologies provided herein are useful for treating a cancer including, but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In certain embodiments, the cancer is breast cancer. In certain embodiments, the cancer is skin cancer. In certain embodiments, the cancer is melanoma. In certain embodiments, the cancer is lung cancer. In certain embodiments, the cancer is kidney cancer. In certain embodiments, the cancer is liver cancer. In certain embodiments, the cancer is pancreatic cancer. In certain embodiments, the cancer is colorectal cancer. In certain embodiments, the cancer is bladder cancer. In certain embodiments, the cancer is lymphoma. In certain embodiments, the cancer is prostate cancer. In certain embodiments, the cancer is thyroid cancer. In certain embodiments, the cancer is brain cancer. In certain embodiments, the cancer is stomach cancer. In certain embodiments, the cancer is esophageal cancer.

In some embodiments, technologies provided herein are useful in treating adenocarcinoma, adrenal gland cancer, anal cancer, angiosarcoma, appendix cancer, bile duct cancer, bladder cancer, bone cancer, brain cancer, breast cancer, bronchus cancer, carcinoid tumor, cardiac tumor, cervical cancer, choriocarcinoma, chordoma, colorectal cancer, connective tissue cancer, craniopharyngioma, ductal carcinoma in situ, endotheliosarcoma, endometrial cancer, ependymoma, epithelial carcinoma, esophageal cancer, Ewing's sarcoma, eye cancer, familiar hypereosinophilia, gall bladder cancer, gastric cancer, gastrointestinal carcinoid tumor, gastrointestinal stromal tumor (GIST), germ cell cancer, head and neck cancer, hemangioblastoma, histiocytosis, Hodgkin lymphoma, hypopharynx cancer, inflammatory myofibroblastic tumors, intraepithelial neoplasms, immunocytic amyloidosis, Kaposi sarcoma, kidney cancer, liver cancer, lung cancer, leiomyosarcoma (LMS), melanoma, midline tract carcinoma, multiple endocrine neoplasia syndrome, muscle cancer, mesothelioma, myeloproliferative disorder (MPD), nasopharynx cancer, neuroblastoma, neurofibroma, neuroendocrine cancer, non-Hodgkin lymphoma, osteosarcoma, ovarian cancer, pancreatic cancer, paraneoplastic syndromes, parathyroid cancer, papillary adenocarcinoma, penile cancer, pharyngeal cancer, pheochromocytoma, pinealoma, pituitary cancer, pleuropulmonary blastoma, primitive neuroectodermal tumor (PNT), plasma cell neoplasia, prostate cancer, rectal cancer, retinoblastoma, rhabdomyosarcoma, salivary gland cancer, sebaceous gland carcinoma, skin cancer, small bowel cancer, small intestine cancer, soft tissue sarcoma, stomach cancer, sweat gland carcinoma, synovioma, testicular cancer, thymic cancer, thyroid cancer, urethral cancer, uterine cancer, vaginal cancer, vascular cancer, vulvar cancer, or a combination thereof.

In some embodiments, a method provided herein may comprise administering to a target site (e.g., as described herein) in a subject an immunomodulatory composition and, optionally, monitoring the subject for risk or incidence of tumor growth, regrowth and/or tumor outgrowth in the subject after the administration, e.g., every 3 months or longer after the administration, including, e.g., every 6 months, every 9 months, every year, or longer. When the subject is determined to have risk or incidence of tumor growth, regrowth, outgrowth, and/or recurrence based on the monitoring report, in some embodiments, a subject can be administered with a second composition (e.g., as described herein) and/or a different treatment regimen (e.g., chemotherapy).

In some embodiments, technologies provided herein may be useful for treating subjects who are suffering from metastatic cancer. For example, in some embodiments, a method provided herein may comprise administering to a target site (e.g., as described herein) in a subject suffering from one or more metastases who has optionally undergone a tumor resection (e.g., surgical resection of a primary tumor) and, optionally, monitoring at least one metastatic site in the subject after the administration, e.g., every 3 months or longer after the administration, including, e.g., every 6 months, every 9 months, every year, or longer. Based on results of the monitoring report, in some embodiments, a subject can be administered with a second composition (e.g., as described herein) and/or a different treatment regimen (e.g., chemotherapy).

In certain embodiments, technologies provided herein do not comprise administering an immunomodulatory composition in association with tumor resection. In certain embodiments, technologies provided herein do comprise administering an immunomodulatory composition in association with tumor resection. In certain embodiments, technologies provided herein comprise administering an immunomodulatory composition to tumor site following tumor resection.

In some embodiments, it will be also appreciated that compositions described herein can be administered in combination with one or more additional pharmaceutical agents. For example, in some embodiments, compositions can be administered in combination with additional pharmaceutical agents that reduce and/or modify their metabolism, inhibit their excretion, and/or modify their distribution within the body. It will also be appreciated that, in some embodiments, the additional therapy employed may achieve a desired effect for the same disorder, and/or it may achieve different effects. In certain embodiments, an additional pharmaceutical agent is not adoptively transferred cells. In certain embodiments, an additional pharmaceutical agent is not T cells. In certain embodiments, an additional pharmaceutical agent is administered multiple days or weeks after administration of a composition described herein.

In certain embodiments, a subject being treated is a mammal. In certain embodiments, a subject is a human. In certain embodiments, a subject is a tumor resection human subject. In certain embodiments, a subject is a human subject that is not amenable to tumor resection surgery. In certain embodiments, a subject is a human patient who has received neoadjuvant (pre-operative) therapy. In certain embodiments, a subject is a human patient who has not received neoadjuvant therapy. In certain embodiments, a subject is a human patient who has received neoadjuvant (pre-operative) chemotherapy. In certain embodiments, a subject is a human patient who has not received neoadjuvant (pre-operative) chemotherapy. In certain embodiments, a subject is a human patient who has received neoadjuvant radiation therapy. In certain embodiments, a subject is a human patient who not has received neoadjuvant radiation therapy. In certain embodiments, a subject is a human patient who has received neoadjuvant chemotherapy and radiation therapy. In certain embodiments, a subject is a human patient who has not received neoadjuvant chemotherapy or radiation therapy. In certain embodiments, a subject is a human patient who has received and/or is receiving neoadjuvant molecular targeted therapy. In some embodiments, a subject is receiving, has received, or will receive immune checkpoint blockade therapy. In certain embodiments, a subject is receiving an immune checkpoint blockade therapy. In certain embodiments, a subject is a human patient who has received and/or is receiving a molecular targeted therapy (e.g., therapies such as those described as neoadjuvants and/or adjuvants) as the sole therapeutic intervention (e.g., a subject for whom surgical resection is not a viable option). In some embodiments, a subject is receiving, has received, or will receive certain other cancer therapeutics (e.g., including but not limited to, costimulation, oncolytic virus, CAR T cells, transgenic TCRs, TILs, vaccines, BiTE, ADC, cytokines, modulators of innate immunity, or any combination of these). In certain embodiments, a subject is a human patient who has received and/or is receiving neoadjuvant immunotherapy, including immune checkpoint blockade (e.g., anti-CTLA-4, anti-PD-1, and/or anti-PD-L1). In certain embodiments, a subject is a human patient who has not received and/or will not receive neoadjuvant immunotherapy, including immune checkpoint blockade (e.g., anti-CTLA-4, anti-PD-1, and/or anti-PD-L1). In certain embodiments, a subject is a human patient whose tumor has not and/or will not objectively responded to neoadjuvant therapy (as defined by Response Evaluation Criteria in Solid Tumors (RECIST) or immune-related Response Criteria (irRC)) (e.g., stable disease, progressive disease). In certain embodiments, a subject is a human patient whose target lesion has objectively responded and/or is objectively responding to neoadjuvant therapy (e.g., partial response, complete response). Non-target lesions may exhibit an incomplete response, stable disease, or progressive disease. In certain embodiments, a subject is a human patient who would be eligible to receive immunotherapy in an adjuvant (post-operative) setting. In certain embodiments, a subject is a domesticated animal, such as a dog, cat, cow, pig, horse, sheep, or goat. In certain embodiments, a subject is a companion animal such as a dog or cat. In certain embodiments, a subject is a livestock animal such as a cow, pig, horse, sheep, or goat. In certain embodiments, a subject is a zoo animal. In another embodiment, a subject is a research animal, such as a rodent, pig, dog, or non-human primate. In certain embodiments, a subject is a non-human transgenic animal such as a transgenic mouse or transgenic pig.

### V. Kits

The present disclosure provides kits that find use in practicing technologies as provided herein. In some embodiments, a kit comprises a composition or a pharmaceutical composition described herein and a container (e.g., a vial, ampule, bottle, syringe, and/or dispenser package, or other suitable container). In some embodiments, one or more component(s) of a composition or a pharmaceutical composition described herein are separately provided in one or more containers. For example, individual components of a composition (e.g., ones described herein) may be, in some embodiments, provided in separate containers. In some such embodiments, individual components of a biomaterial (e.g., ones described herein, for example, but not limited to hyaluronic acid, chitosan, poloxamer, etc.) may be each provided independently as dry lyophilized powder, dry particles, or a liquid. In some embodiments, individual components such as delivery agents and/or technologies (e.g., syringes, bags, etc., or component(s) thereof) may be provided as a single and/or multiple use item in a container. In some embodiments, individual components of a composition may be provided as a single mixture in a container. In some such embodiments, a single mixture may be provided as dry lyophilized powder, dry particles, or a liquid (e.g., a homogenous liquid).

In some embodiments, a composition described herein may be provided as a pre-formed polymer network biomaterial (incorporated with a modulator of myeloid-derived suppressive cell function) in a container. In some embodiments, such a pre-formed polymer network biomaterial (e.g., a hydrogel) may be provided in a dried state. In some embodiments, such a pre-formed polymer network biomaterial (in a form of a viscous polymer solution) may be provided in a container.

In some embodiments, provided kits may optionally include a container comprising a pharmaceutical excipient for dilution or suspension of a composition or pharmaceutical composition described herein. In some embodiments, provided kits may include a container comprising an aqueous solution. In some embodiments, provided kits may include a container comprising a buffered solution.

In some embodiments, provided kits may comprise a payload such as a therapeutic agent described herein. For example, in some embodiments, a payload may be provided in a separate container such that it can be added to a biomaterial preparation liquid mixture (e.g., as described herein) prior to administration to a subject. In some embodiments, a payload may be incorporated in a biomaterial preparation described herein.

In certain embodiments, a kit described herein further includes instructions for practicing methods described herein. A kit described herein may also include information as required by a regulatory agency such as the U.S. Food and Drug Administration (FDA). In certain embodiments, information included in kits provided herein is prescribing information, e.g., for treatment for cancer. Instructions may be present in kits in a variety of forms, one or more of which may be present in the kits. One form in which these instructions may be present is as printed information on a suitable medium or substrate, e.g., a piece or pieces of paper on which the information is printed, in the packaging of kits, in a package insert, etc. Yet another means may be a computer readable medium, e.g., diskette, CD, USB drive, etc., on which instructional information has been recorded. Yet another means that may be present is a website address which may be used via the internet to access instructional information. Any convenient means may be present in the kits.

Other features of the invention will become apparent in the course of the following description of exemplary embodiments, which are given for illustration of the invention and are not intended to be limiting thereof.

### EXAMPLES

In order that the invention described herein may be more fully understood, the following examples are set forth. It should be understood that these examples are for illustrative purposes only and are not to be construed as limiting this invention in any manner.

### Example 1. Preparation and Uses of Exemplary Composition Described Herein Comprising a TLR7/8 Agonist for In Vivo Cancer Treatment

The present Example describes administration of a composition comprising an immunomodulatory composition at target site that is or includes a lymph node resection site. In some embodiments, the target site additionally is or includes a primary tumor resection site. In some embodiments, the immunomodulatory composition comprises a biomaterial preparation and an immunomodulatory payload. In some embodiments, such an immunomodulatory payload is or comprises a TLR7/8 agonist. In some embodiments, such a biomaterial preparation is a thermo-responsive polymer solution that forms a hydrogel at physiological temperature.

In some embodiments, in vivo studies utilized breast cancer cell lines grown in vitro. In some embodiments, prior to implantation, breast cancer cells were cultured in a medium containing fetal bovine serum and an appropriate selective agent. In some embodiments, cells were cultured at physiological temperature, with known humidity, oxygen, and CO₂ concentrations.

In some embodiments, 4T1-Luc2 breast cancer cells were cultured in RPMI-1640 medium, with 10% fetal bovine serum (FBS) and 1% penicillin/streptomycin. In some embodiments, cells were cultured in a 37° C. humidified incubator, with 5% CO₂.

In some embodiments, in vivo animal studies are performed in mice. In some embodiments, mice are inoculated orthotopically with breast cancer cells in a mammary fat pad. In some embodiments, surgery is performed at an appropriate time following inoculations (e.g., at approximately day 10 after tumor inoculation). In some embodiments, concurrent with lymph node resection and primary tumor resection, a composition as described herein comprising a biomaterial preparation and an immunomodulatory payload is administered at a target site that is or includes the lymph node resection site. In some embodiments, a composition containing a biomaterial preparation without an immunomodulatory payload is administered at the target site that is or includes the lymph node resection site to act as a relative negative control. In some embodiments, prolonged survival benefits are observed upon extended local release of the immunomodulatory payload.

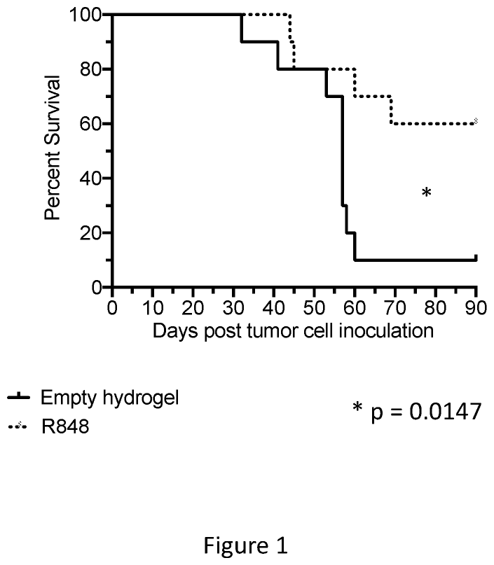

In one embodiment, in vivo animal studies were performed in 6-8 week old female BALB/cJ mice. Mice were inoculated orthotopically with 10⁵ 4T1-Luc2 breast cancer cells in their fourth mammary fat pad. Mice were size-matched and randomly assigned to treatment groups, and surgery was performed at day 10 after tumor inoculation. For primary tumor resection and proximal inguinal lymph node resection, mice were anesthetized with 2% isoflurane, and the primary tumor and proximal inguinal lymph node were resected. Concurrently, a composition described herein comprising biomaterial preparation (e.g., containing a thermo-responsive polymer solution that forms a hydrogel at physiological temperature (e.g., a biomaterial preparation including 9-11% w/w poloxamer and 2.0-2.5% w/w hyaluronic acid)) loaded with an immunomodulatory payload (e.g., a TLR7/8 agonist (e.g. resiquimod (R848), for example in some embodiments at a dose of 0.2 mg/mouse) was implanted at a target site that is or includes the lymph node resection site. Empty biomaterial was used as a negative control. Prolonged survival benefit was observed upon extended local release of R848 (FIG. 1). Survival curves were analyzed using Kaplan-Meier survival analysis with the log-rank (Mantel-Cox) test. Significant differences were observed in animals treated with the composition containing R848 when compared to negative control (p=0.0147).

### Example 2. Preparation and Uses of Exemplary Composition Described Herein Comprising a TLR7/8 Agonist for In Vivo Cancer Treatment

The present Example describes administration of a composition comprising an immunomodulatory composition at target site that is or includes a lymph node resection site. In some embodiments, the target site is not or does not include a primary tumor resection site. In some embodiments, the immunomodulatory composition comprises a biomaterial preparation and an immunomodulatory payload. In some embodiments, such an immunomodulatory payload is or comprises a TLR7/8 agonist. In some embodiments, such a biomaterial preparation is a thermo-responsive polymer solution that forms a hydrogel at physiological temperature.

In some embodiments, in vivo studies utilize breast cancer cell lines grown in vitro. In some embodiments, prior to implantation, breast cancer cells are cultured in a medium containing fetal bovine serum and an appropriate selective agent. In some embodiments, cells are cultured at physiological temperature, with known humidity, oxygen, and CO2 concentrations.

In some embodiments, 4T1-Luc2 breast cancer cells are cultured in RPMI-1640 medium, with 10% fetal bovine serum (FBS) and 1% penicillin/streptomycin. In some embodiments, cells are cultured in a 37° C. humidified incubator, with 5% CO2.

In some embodiments, in vivo animal studies are performed in mice. In some embodiments, mice are inoculated orthotopically with breast cancer cells in a mammary fat pad. In some embodiments, surgery is performed at an appropriate time following inoculations (e.g., at approximately day 10 after tumor inoculation). In some embodiments, concurrent with lymph node resection and primary tumor resection, a composition as described herein comprising a biomaterial preparation and an immunomodulatory payload is administered at a target site that is or includes the lymph node resection site. In some embodiments, the target site is not or does not include a primary tumor resection site. In some embodiments, a composition containing a biomaterial preparation without an immunomodulatory payload is administered at the target site that is or includes the lymph node resection site to act as a relative negative control. In some embodiments, prolonged survival benefits are observed upon extended local release of the immunomodulatory payload.

In one embodiment, in vivo animal studies are performed in 6-8 week old female BALB/cJ mice. Mice are inoculated orthotopically with 10⁵ 4T1-Luc2 breast cancer cells in their fourth mammary fat pad. Mice are size-matched and randomly assigned to treatment groups, and surgery is performed at day 10 after tumor inoculation. For primary tumor resection and proximal lymph node resection, mice are anesthetized with 2% isoflurane, and the primary tumor and proximal lymph node are resected. Concurrently, a composition described herein comprising biomaterial preparation (e.g., containing a thermo-responsive polymer solution that forms a hydrogel at physiological temperature) loaded with an immunomodulatory payload (e.g., a TLR7/8 agonist (e.g. resiquimod (R848)) is implanted at a target site that is or includes the lymph node resection site and that is not or does not include a primary tumor resection site. Empty biomaterial is used as a negative control. Prolonged survival benefit is expected to be observed upon extended local release of R848. Survival curves are analyzed using Kaplan-Meier survival analysis with the log-rank (Mantel-Cox) test. Significant differences are expected to be observed in animals treated with the composition containing R848 when compared to negative control.

### Example 3. Preparation and Uses of Exemplary Composition Described Herein for In Vivo Cancer Treatment

The present Example describes administration of a composition comprising an immunomodulatory composition at target site that is or includes a lymph node resection site. In some embodiments, the target site is not or does not include a primary tumor resection site. In some embodiments, the immunomodulatory composition comprises a biomaterial preparation and an immunomodulatory payload. In some embodiments, such an immunomodulatory payload is or comprises a modulator of innate immunity, a modulator of myeloid cell function, a modulator of adaptive immunity, a modulator of inflammation, a TLR7/8 agonist (e.g., resiquimod), a COX inhibitor (e.g., a COX-1 inhibitor and/or a COX-2 inhibitor), a NSAID (e.g., ketorolac), an angiotensin II receptor inhibitor (e.g., valsartan), a CXCR4 receptor antagonist (e.g., plerixafor), an immunomodulatory cytokine (e.g., IL-2 or IL-12), or a combination thereof. In some embodiments, such a biomaterial preparation is or comprises a thermo-responsive polymer (e.g., a poloxamer), a carbohydrate polymer (e.g., hyaluronic acid and/or chitosan or a modified chitosan, or a combination thereof.

In some embodiments, in vivo studies utilize breast cancer cell lines grown in vitro. In some embodiments, prior to implantation, breast cancer cells are cultured in a medium containing fetal bovine serum and an appropriate selective agent. In some embodiments, cells are cultured at physiological temperature, with known humidity, oxygen, and CO₂ concentrations.

In some embodiments, 4T1-Luc2 breast cancer cells are cultured in RPMI-1640 medium, with 10% fetal bovine serum (FBS) and 1% penicillin/streptomycin. In some embodiments, cells are cultured in a 37° C. humidified incubator, with 5% CO₂.

In some embodiments, in vivo animal studies are performed in mice. In some embodiments, mice are inoculated orthotopically with breast cancer cells in a mammary fat pad. In some embodiments, surgery is performed at an appropriate time following inoculations (e.g., at approximately day 10 after tumor inoculation). In some embodiments, concurrent with lymph node resection, a composition as described herein comprising a biomaterial preparation and an immunomodulatory payload is administered at a target site that is or includes the lymph node resection site. In some embodiments, lymph node resection occurs during a tumor resection surgery. In some embodiments, lymph node resection occurs without a tumor resection surgery. In some embodiments, the target site is not or does not include a primary tumor resection site. In some embodiments, a composition containing a biomaterial preparation without an immunomodulatory payload is administered at the target site that is or includes the lymph node resection site to act as a relative negative control. In some embodiments, a composition containing an immunomodulatory payload without a biomaterial preparation is administered at the target site that is or includes the lymph node resection site to act as a relative negative control. In some embodiments, prolonged survival benefits are observed upon extended local release of the immunomodulatory payload.

In one embodiment, in vivo animal studies are performed in 6-8 week old female BALB/cJ mice. Mice are inoculated orthotopically with 10⁵ 4T1-Luc2 breast cancer cells in their fourth mammary fat pad. Mice are size-matched and randomly assigned to treatment groups, and surgery is performed at day 10 after tumor inoculation. For lymph node resection, mice are anesthetized with 2% isoflurane, and the lymph node is resected. Lymph node resection may occur during a tumor resection surgery; or without a tumor resection surgery. Concurrently, a composition described herein comprising biomaterial preparation loaded with an immunomodulatory payload is implanted at a target site that is or includes the lymph node resection site and that is not or does not include a primary tumor resection site. Empty biomaterial and/or immunomodulatory payload alone is used as a negative control. Prolonged survival benefit is expected to be observed upon extended local release of the immunomodulatory payload. Survival curves are analyzed using Kaplan-Meier survival analysis with the log-rank (Mantel-Cox) test. Significant differences are expected to be observed in animals treated with the composition containing the biomaterial preparation and the immunomodulatory payload when compared to negative control.

### Equivalents and Scope

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference for purposes described herein. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The entire content of International Patent Publication No. WO 2018/045058 (e.g., compositions, devices, methods of preparation, methods of use, and kits) is incorporated herein by reference for the purposes described herein.

What is claimed is:

1. A method of treating cancer, comprising a step of administering to a target site in a cancer subject, a composition comprising an effective amount of an immunomodulatory composition, wherein the target site is or comprises a lymph node dissection site.

2. The method of claim 1, wherein the immunomodulatory composition is characterized by its ability to induce an innate immune response.

3. The method of claim 1, wherein the immunomodulatory composition is characterized by its ability to inhibit an immunosuppressive inflammation response.

4. The method of claim 1, wherein the immunomodulatory composition is or comprises a biomaterial preparation.

5. The method of claim 1, wherein the immunomodulatory composition comprises a biomaterial preparation and at least one immunomodulatory payload.

6. The method of claim 4 or 5, wherein the biomaterial preparation comprises at least one polymer (including, e.g., at least two polymers).

7. The method of claim 5, wherein the immunomodulatory payload is or comprises a modulator of innate immunity.

8. The method of claim 5, wherein the immunomodulatory payload is or comprises a modulator of myeloid cell function.

9. The method of claim 5, wherein the immunomodulatory payload is or comprises a modulator of adaptive immunity.

10. The method of claim 5, wherein the immunomodulatory payload is or comprises a modulator of inflammation.

11. The method of any one of claims 5-7, wherein the immunomodulatory payload is or comprises a TLR7/8 agonist.

12. The method of claim 11, wherein the immunomodulatory payload is or comprises resiquimod.

13. The method of claim 10, wherein the immunomodulatory payload is or comprises a COX inhibitor (e.g., a COX-1 inhibitor and/or a COX-2 inhibitor).

14. The method of claim 10, wherein the immunomodulatory payload is or comprises an NSAID, e.g., ketorolac.

15. The method of claim 5, wherein the immunomodulatory payload is or comprises an angiotensin II receptor inhibitor, e.g., valsartan.

16. The method of claim 5, wherein the immunomodulatory payload is or comprises a CXCR4 receptor antagonist, e.g., plerixafor.

17. The method of claim 5, wherein the immunomodulatory payload is or comprises an immunomodulatory cytokine, e.g., IL-2 or IL-12.

18. The method of any one of claims 1-17, wherein the cancer subject is a tumor resection subject.

19. The method of any one of claims 1-18, comprising, prior to the step of administering, removing at least one lymph node that is proximal to a tumor in the cancer subject.

20. The method of claim 19, wherein the removal of at least one lymph node is performed intraoperatively during a tumor resection surgery.

21. The method of claim 19, wherein the removal of at least one lymph node is performed in a different operation from a tumor resection surgery.

22. The method of claim 20 or 21, further comprising intraoperative administration of a second immunomodulatory composition at the tumor resection site.

23. The method of claim 22, wherein the second immunomodulatory composition administered at the tumor resection site is the same as the immunomodulatory composition administered at the lymph node dissection site.

24. The method of claim 22, wherein the second immunomodulatory composition administered at the tumor resection site is different from the immunomodulatory composition administered at the lymph node dissection site.

25. The method of any of claims 18-24, wherein tumor resection surgery comprises removal of at least a portion of an organ comprising a tumor.

26. The method of claim 25, wherein the tumor resection surgery further comprises anastomosis after the removal of at least a portion of the organ comprising the tumor.

27. The method of any one of claims 1-26, wherein the lymph node is or comprises: a sentinel lymph node, a draining lymph node, an axillary lymph node, an inguinal lymph node, a femoral lymph node, a facial lymph node, a neck lymph node, a cervical lymph node, a supraclavicular lymph node, a subclavian lymph node, a pectoral lymph node, a mediastinal lymph node, a pelvic lymph node, a mesenteric lymph node, and/or a retroperitoneal lymph node.

28. The method of claim 27, wherein the lymph node is a sentinel lymph node.

29. The method of claim 27, wherein the lymph node is a draining lymph node.

30. The method of any one of claims 1-29, comprising intraoperative administration of the composition to the target site in a cancer subject undergoing a lymph node dissection surgery.

31. The method of any one of claims 1-30, wherein the lymph node dissection surgery further comprises anastomosis.

32. The method of any one of claims 1-31, wherein the cancer being treated is at least one of: a carcinoma, a sarcoma, a germ cell tumor, a blastoma, a lymphoma, a skin cancer, a melanoma, a pharyngeal head and neck cancer, a thyroid cancer, a brain cancer, a bladder cancer, a gastrointestinal tract cancer (e.g., a stomach cancer), a thoracic cancer, a lung cancer, a breast cancer, a colorectal cancer, a genitourinary cancer, a kidney cancer, a prostate cancer, a gynecologic cancer, a testicular cancer, an ovarian cancer, and or an uterine cancer.

33. The method of any of claims 4-32, wherein the biomaterial preparation comprises a thermo-responsive polymer.

34. The method of claim 33, wherein the thermo-responsive polymer is or comprises a poloxamer.

35. The method of any one of claims 4-34, wherein the biomaterial preparation comprises a carbohydrate polymer.

36. The method of claim 35, wherein the carbohydrate polymer comprises hyaluronic acid and/or chitosan or a modified chitosan.

37. The method of any one of claims 4-36, wherein the biomaterial preparation comprises a poloxamer at 7-12.5% (w/w), and one or both of a hyaluronic acid at 0.5-7% (w/w) and chitosan or a modified chitosan at 0.5-7% (w/w).

38. The method of any one of claims 1-36, wherein the method reduces the likelihood of developing one or more symptoms associated with lymphedema and/or lymphocele as compared to a lymph node dissection without administration of an immunomodulatory composition.

39. The method of any one of claims 1-37, wherein the method reduces the likelihood of developing one or more metastatic lesions as compared to a lymph node dissection without administration of an immunomodulatory composition.

40. The method of any one of claims 1-38, wherein the administration at the lymph node dissection site is performed by implantation.

41. The method of any one of claims 1-38, wherein the administration at the lymph node dissection site is performed by injection.

42. In a method of treating cancer by intraoperative administration of a combination of a biomaterial preparation and an immunomodulatory payload to a subject suffering from cancer, the improvement that comprises:
administering the combination at a lymph node dissection site rather than or in addition to at a tumor resection site.
